(19) United States
(12) Patent Application Publication
(10) Pub. No.: US 2023/0357159 A1
(43) Pub. Date: Nov. 9, 2023

(54) QUINOLINE COMPOUNDS AS SELECTIVE AND/OR DUAL MODULATORS OF BILE ACID RECEPTORS AND LEUKOTRIENE CYSTEINYL RECEPTORS

(71) Applicant: PRECISION BIO-THERAPEUTICS S.R.L., Perugia (IT)

(72) Inventors: Angela Zampella, Perugia (IT); Stefano Fiorucci, Perugia (IT)

(21) Appl. No.: 18/019,711

(22) PCT Filed: Aug. 4, 2021
(86) PCT No.: PCT/IB2021/057131

§ 371 Date: Feb. 3, 2023

(30) Foreign Application Priority Data
Aug. 4, 2020 (IT) 102020000019210

(51) Int. Cl. A61P 1/16; A61P 3/00; C07D 215/14

(52) U.S. Cl. CPC C07D 215/14; A61P 1/16; A61P 3/00

(57) ABSTRACT

The present invention relates to compounds of formula (I), their pharmaceutical compositions and uses, in particular for the treatment and/or prevention of diseases mediated by bile acid receptors, FXR and GPBAR1, and cysteinyl leukotriene receptors (CysLTR).

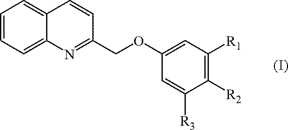

### CROSS-REFERENCE TO RELATED APPLICATIONS

This patent application claims priority from Italian patent application no. 102020000019210 filed on Aug. 4, 2020, the entire disclosure of which is incorporated herein by reference.

### TECHNICAL SECTOR

The present invention relates to quinoline derivatives and uses thereof that can simultaneously modulate bile acid receptors, FXR and GPBAR1, and cysteinyl leukotriene receptors (CysLTR) and their use in the treatment and/or prevention of diseases mediated by the latter.

### STATE OF THE PRIOR ART

The strategy of identifying small molecules capable of acting simultaneously on multiple targets is widely recognised as useful in identifying new pharmacological approaches to multifactorial diseases such as chronic inflammatory disorders, including non-alcoholic steatohepatitis, a highly prevalent inflammatory liver disease, metabolic syndrome, and cancer.

This study starts from our recent observation that REV5901, a cysteinyl-leukotriene receptor antagonist is able to modulate GPBAR1 with interesting anti-inflammatory activity in an animal model of intestinal inflammation, and that Zafirlukast, a well-known receptor antagonist for CysLTs has weak activity against FXR (S. Schierle, et al. Anti-Inflammatory Potency of Zafirlukast by Designed Polypharmacology, J Med Chem 61(13) (2018) 5758-5764).

Leukotrienes are a large family of lipid mediators that are generated from arachidonic acid through an enzymatic cascade and function as mediators of inflammation. Among the leukotrienes, the cysteinyl leukotrienes (CysLTs), which comprise LTC4, LTD4 and LTE4, act on cells by binding to a family of G-protein-associated transmembrane proteins (CysLTR), expressed on many pro-inflammatory cells such as neutrophils and eosinophils, mast cells and monocytes/macrophages. The activation of these receptors by endogenous lipid mediators plays a significant role in the inflammatory response resulting in microvascular permeability, leukocyte trafficking, secretion of chemokines and cytokines and tissue repair (fibrosis). It is well known that cysteinyl leukotriene receptors mediate bronchoconstriction, pulmonary mucus secretion and oedema, and consequently their antagonists are validated drugs in the treatment of asthma and more generally in the pharmacological approach to pulmonary disorders. Cysteinyl leukotrienes are implicated in many other diseases such as cardiovascular disorders, cancer, atopic dermatitis, rheumatoid arthritis, Crohn’s disease, in the pathogenesis of fulminant hepatitis as well as in liver cholestasis, fibrosis and cirrhosis (Capra V.et al. Cysteinyl-leukotrienes and their receptors in asthma and other inflammatory diseases: critical update and emerging trends. (Med Res Rev. 2007 Jul; 27(4):469-527).

Highly expressed in enterohepatic tissues (liver and intestine), FXR regulates bile acid homeostasis and some metabolic pathways, including lipid and glucose metabolism. FXR agonists have proved useful in the pharmacological approach to metabolic disorders such as cholestasis, type 2 diabetes, liver fibrosis and non-alcoholic fatty liver syndrome (NAFLD). Furthermore, FXR plays an important role in the kidney, the cardiovascular system and in tumour genesis (Renga et al. PHASEB J. 2012, 26, 3021-3031).

GPBAR1 is highly expressed in the liver and intestine, but also in muscles, adipose tissue, macrophages and endothelial cells. In the muscle and in the brown adipose tissue, GPBAR1 increases the energy expenditure and the oxygen consumption (Watanabe et al. Nature of 2006, 439, 484). In entero-endocrine L-cells, the GPBAR1 activation stimulates the secretion of glucagon-like peptide (GLP-1), thereby regulating blood glucose levels, gastrointestinal motility and appetite (Thomas et al. Cell. Metab. 2009, 10, 167).

GPBAR1 appears to be relevant in the regulation of the inflammatory process and of the immune function. Many cells of the innate immunity express this receptor, such as monocytes, macrophages, the NKT cells and the dendritic cells, and mutations in this receptor are associated with an increased risk of developing primary sclerosing cholangitis and ulcerative colitis.

### OBJECT OF THE INVENTION

The object of the present invention is to identify novel compounds that can selectively or simultaneously modulate bile acid receptors, FXR and GPBAR1, and cysteinyl leukotriene receptors (CysLTR).

Such an object is achieved by the present invention, with respect to the compounds of Formula (I) according to claim 1, to their use according to claims 8 and 9 and to a composition thereof according to claim 10. The preferred embodiments are indicated in the dependent claims.

### BRIEF DESCRIPTION OF THE DRAWINGS

The present invention will now be described in detail by reference to the figures in the accompanying drawings, in which:

FIG. 1 shows the AST values in mice in which acute hepatitis is induced with acetaminophen and subsequently treated with CHIN117.

FIG. 2 shows the ALT values in mice in which acute hepatitis is induced with acetaminophen and subsequently treated with CHIN117.

FIG. 3 shows the white blood cell (WBC) values in mice in which acute hepatitis is induced with acetaminophen and subsequently treated with CHIN117.

FIG. 4 shows the results of CHIN117 administration in a murine model simulating NAFLD: (A) the change in body weight (%) was assessed weekly; (B) the area under the curve (AUC) of body weight; (C) the brown adipose tissue temperature (BAT) (°C); (D) the glucose levels in response to oral glucose tolerance test (OGTT); (E) AUC of the OGTT; (G) plasma levels of AST (U/L) and ALT (U/L); (H) the plasma levels of cholesterol, triglycerides, high density lipoproteins (HDLs), and low density lipoproteins (LDLs) (mg/100 mL). The results are the mean ± SEM of 8-12 mice per group. *p≤ 0.05.

FIG. 5 shows the results of CHIN117 administration in a C57BL/6 murine model fed an HFD-F diet for 8 weeks: (A) Haematoxylin and eosin (H & E) staining on murine liver tissue (4x-10x). The disease severity was assessed by calculating (B) the steatosis score (NAS); (C) the body mass index (BMI); (D) the eWAT weight; (E) the eWAT weight/body weight ratio (F) the BAT weight (g) (G) the BAT weight/body weight ratio (H) the liver weight (I) the liver weight/body weight ratio. The results are the mean ± SEM of 8-12 mice per group. *p≤ 0.05.

### PREFERRED EMBODIMENT OF THE INVENTION

The following paragraphs provide the chemical characteristics of the compounds according to the invention and are intended to be applied uniformly to the entire description and all claims unless a definition providing a broader definition is expressly stated otherwise.

The term “alkyl”, as used herein, refers to saturated aliphatic hydrocarbons. This term includes linear (unbranched) or branched chains.

Non-limiting examples of alkyl groups according to the invention are, for example, methyl, ethyl, propyl, isopropyl, n-butyl, isobutyl, tert-butyl, n-pentyl, isopentyl, n-hexyl and the like.

The term “hydroxyalkyl”, as used herein, refers to saturated aliphatic hydrocarbons in which one or more hydrogen atoms are substituted with a hydroxyl group.

Unless otherwise indicated, the term “substituted”, as used herein, means that one or more hydrogen atoms of the above groups are replaced with another non-hydrogen atom, or functional group, provided that the normal valences are maintained and that the substitution results in a stable compound.

Those skilled in the art of organic chemistry will appreciate that many organic compounds can form complexes with solvents in which they are reacted or precipitated or crystallised from. These complexes are known as “solvates”. For example, a complex with water is known as a “hydrate”. The solvates of the compounds of the invention fall within the scope of the invention. The compounds of formula (I) or (Ia) can be easily isolated in association with solvent molecules by crystallisation or evaporation of an appropriate solvent to provide the corresponding solvates.

The compounds of formula (I) or (Ia) can be in crystalline form. In some embodiments, the crystalline forms of the compounds of formula (I) or (Ia) are polymorphic.

The present invention also includes isotopically labelled compounds, which are identical to those given in formula (I) or (Ia), but differ in that one or more atoms are replaced by an atom having an atomic mass or mass number different from the atomic mass or mass number usually found in nature. Examples of isotopes that can be incorporated into the compounds of the invention include isotopes of hydrogen, carbon, nitrogen, and oxygen such as ²H, ³H, ¹¹C, ¹³C, ¹⁴C_{,} ¹⁵N, ¹⁷O_{.}

The compounds of the present invention that contain the above-mentioned isotopes and/or other isotopes of other atoms fall within the scope of protection of the present invention. The isotopically labelled compounds of the present invention, for example those in which radioactive isotopes such as ³H e ¹⁴C are incorporated, are useful in assays of tissue distribution of drug and/or substrate. Tritiated isotopes, i.e. ³H and carbon-14, i.e. ¹⁴C, are particularly preferred due to their ease of preparation and detectability. The isotopes ¹¹C are particularly useful in PET (positron emission tomography). In addition, the substitution with heavier isotopes such as deuterium, i.e. ²H, may provide certain therapeutic advantages resulting from the increased metabolic stability, e.g. increased in vivo half-life or reduced dosing requirements, and may therefore be referred to in certain circumstances. The isotopically labelled compounds of formula (I) or (Ia) of the present invention can generally be prepared by performing the processes described in the following diagrams and/or examples, substituting a non-isotopically labelled reagent for a readily available isotopically labelled reagent.

Some groups/substituents included in the present invention may be present as isomers. Consequently, in some embodiments, the compounds of formula (I) or (Ia) may have axial asymmetries and, correspondingly, may exist in the form of optical isomers such as a form (R), a form (S) and the like. The present invention includes within the scope of protection all such isomers, including racemates, enantiomers and mixtures thereof.

In particular, the scope of protection of the present invention includes all stereoisomeric forms, including enantiomers, diastereoisomers and mixtures thereof, including racemates, and the general reference to the compounds of formula (I) or (Ia) includes all stereoisomeric forms, unless otherwise indicated.

In general, the compounds of the invention should be considered to exclude those compounds (if any) which are chemically very unstable, either by themselves or in water, to be clearly unsuitable for pharmaceutical use by all routes of administration, regardless of the whether it is oral, parenteral or otherwise. Such compounds are known to the skilled chemist.

Finally, the compounds of formula (I) or (Ia) can form salts. In particular, the quinoline ring is capable of forming hydrochloride salts, while the phenolic residues or COOH groups form metal salts.

According to a first aspect of the invention, the compounds of formula (I) are provided:

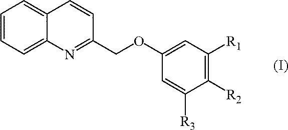

or pharmaceutically acceptable salts or solvates thereof wherein:
R₁ is selected from the group consisting of H, linear or branched C₁₋₆ alkyl optionally substituted with one substituent R₇; linear or branched O-C₃₋₆ alkyl optionally substituted with one substituent R₈;
R₂ is selected from the group consisting of H, C₁₋₆ hydroxyalkyl, linear or branched C₁₋₆ alkyl optionally substituted with one substituent R₇; phenyl optionally substituted with at least one substituent independently selected from the group consisting of H, COOH, COO—C₁₋₆ alkyl, C₁₋₆ hydroxyalkyl and linear or branched C₁₋₆ alkyl optionally substituted with one substituent R₉;
R₃ is selected from the group consisting of H, COOH, COO—C₁₋₆ alkyl, CH₂OH;
provided that:
   when R₂ is H, R₁ or R₃ are not H; or
   when R₁ is linear O-C₃₋₆ alkyl optionally substituted with one substituent R₈, at least one between R₂ and R₃ is not H;
R₄ is selected from the group consisting of H, COOH, COO—C₁₋₆ alkyl, C₁₋₆ hydroxyalkyl;
R₅ is selected from the group consisting of H, COOH, COO—C₁₋₆ alkyl, C₁₋₆ hydroxyalkyl;
R₆ is selected from the group consisting of H, COOH, COO—C₁₋₆ alkyl, C₁₋₆ hydroxyalkyl, OH and linear or branched C₁₋₆ alkoxyl optionally substituted with one substituent R₈;
provided that when one between R₄, R₅ and R₆ is COOH or CH₂OH, at least one of the remaining ones between R₄, R₅ and R₆ is not H,
R₇ is selected from the group consisting of:

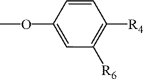

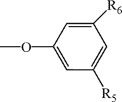

R₈ is selected from the group consisting of OH, COOH, COO—C₁₋₆ alkyl;
R₉ is selected from the group consisting of:

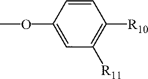

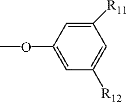

where R₁₀ is selected from the group consisting of H, COOH, COO—C₁₋₆ alkyl, C₁₋₆ hydroxyalkyl;
R₁₁ is selected from the group consisting of H, OH, COOH, COO—C₁₋₆ alkyl, C₁₋₆ hydroxyalkyl;
and R₁₂ is selected from the group consisting of H, COOH, COO—C₁₋₆ alkyl, C₁₋₆ hydroxyalkyl;
excluding the following compounds

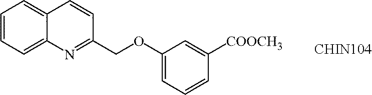 [SMILES: COC(=O)c1cccc(OCc2ccc3ccccc3n2)c1]

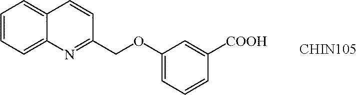 [SMILES: O=C(O)c1cccc(OCc2ccc3ccccc3n2)c1]

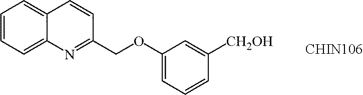 [SMILES: OCc1cccc(OCc2ccc3ccccc3n2)c1]

In a first embodiment, R₁ is selected from the group consisting of H, O-i-propyl, O-n-propyl, O-n-butyl, O-sec-butyl, O-n-pentyl, O-2-methylbutyl, —CH₂—R₇, —O— (CH₂) ₃₋₄—R₈.

In one embodiment, R₂ is a phenyl optionally substituted with at least one substituent independently selected from the group consisting of H, COOH, COO—C₁₋₆ alkyl, C₁₋₆ hydroxyalkyl and linear or branched C₁₋₆ alkyl optionally substituted with one substituent R₉.

In a further embodiment, R₂ is selected from the group consisting of H, CH₂OH, phenyl substituted with two substituents independently selected from the group consisting of H, COOH, COO—C₁₋₆ alkyl, C₁₋₆ hydroxyalkyl, —CH₂—R₉.

### In a Third Embodiment,

R₃ is selected from the group consisting of H, COOH, COOCH₃, CH₂OH;
R₄ is selected from the group consisting of H, COOH, COOCH₃, CH₂OH; and
R₅ is selected from the group consisting of H, COOH, COOCH₃, CH₂OH.

In a further embodiment, the compounds of formula (I) are selected from the group consisting of:

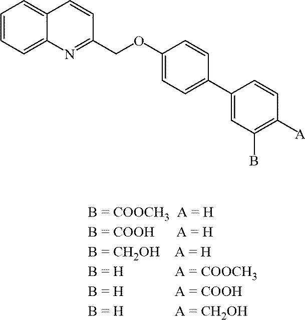

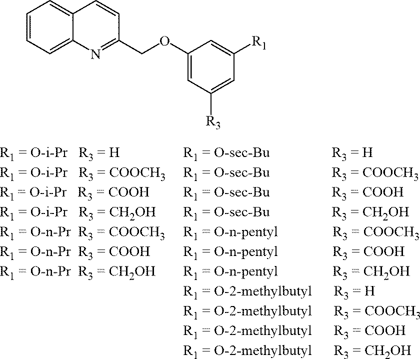

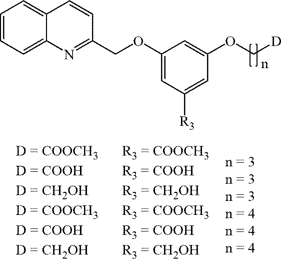

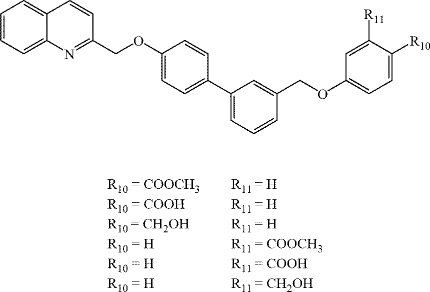

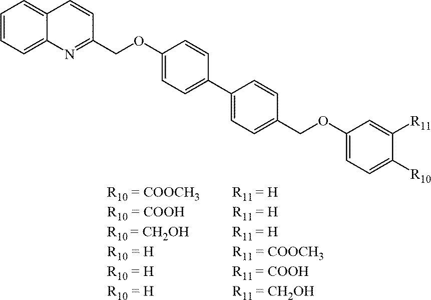

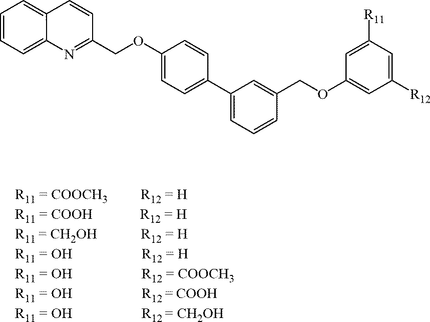

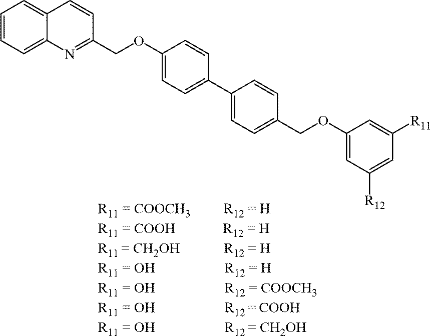

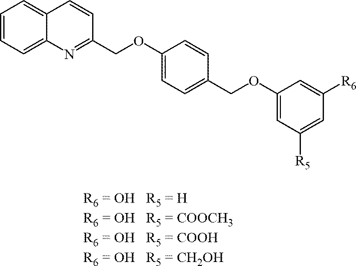

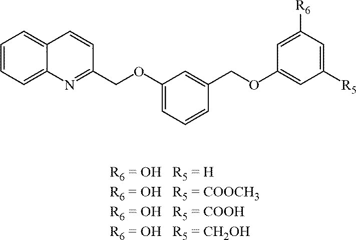

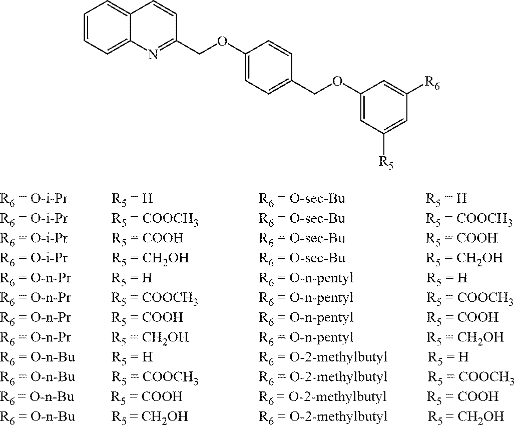

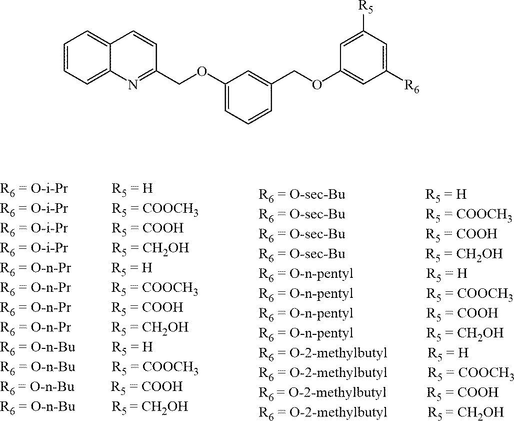

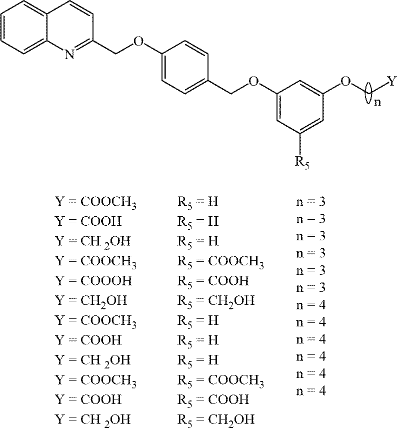

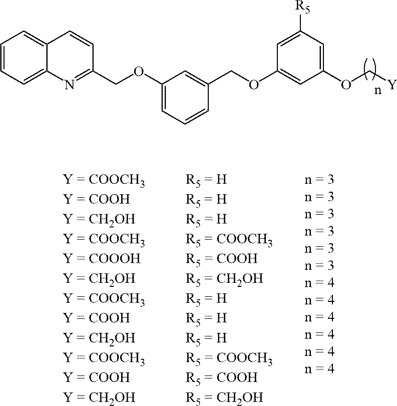

Preferably, the compounds of formula (I) are selected from the group consisting of:

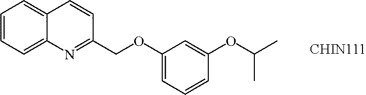 [SMILES: CC(C)Oc1cccc(OCc2ccc3ccccc3n2)c1]

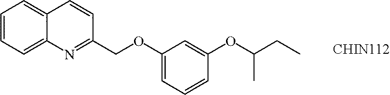 [SMILES: CCC(C)Oc1cccc(OCc2ccc3ccccc3n2)c1]

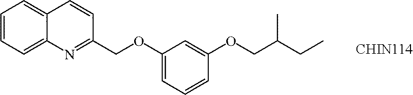 [SMILES: CCC(C)COc1cccc(OCc2ccc3ccccc3n2)c1]

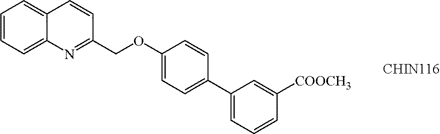 [SMILES: COC(=O)c1cccc(-c2ccc(OCc3ccc4ccccc4n3)cc2)c1]

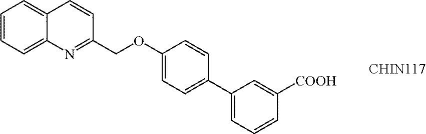 [SMILES: O=C(O)c1cccc(-c2ccc(OCc3ccc4ccccc4n3)cc2)c1]

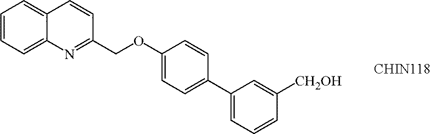 [SMILES: OCc1cccc(-c2ccc(OCc3ccc4ccccc4n3)cc2)c1]

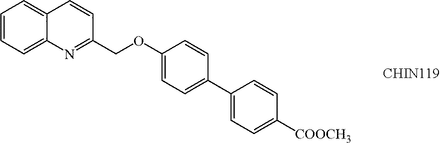 [SMILES: COC(=O)c1ccc(-c2ccc(OCc3ccc4ccccc4n3)cc2)cc1]

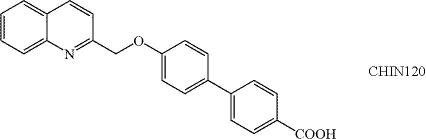 [SMILES: O=C(O)c1ccc(-c2ccc(OCc3ccc4ccccc4n3)cc2)cc1]

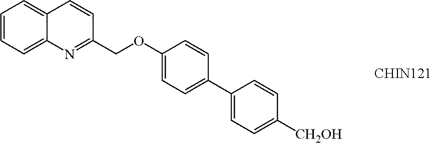 [SMILES: OCc1ccc(-c2ccc(OCc3ccc4ccccc4n3)cc2)cc1]

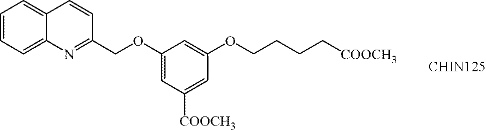 [SMILES: COC(=O)CCCCOc1cc(OCc2ccc3ccccc3n2)cc(C(=O)OC)c1]

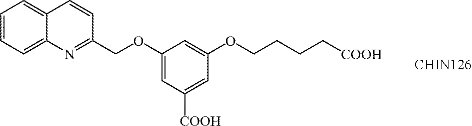 [SMILES: O=C(O)CCCCOc1cc(OCc2ccc3ccccc3n2)cc(C(=O)O)c1]

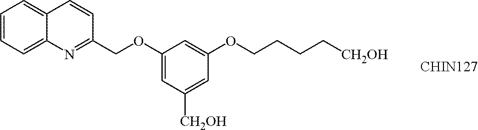 [SMILES: OCCCCCOc1cc(CO)cc(OCc2ccc3ccccc3n2)c1]

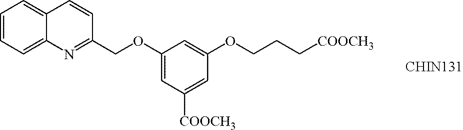 [SMILES: COC(=O)CCCOc1cc(OCc2ccc3ccccc3n2)cc(C(=O)OC)c1]

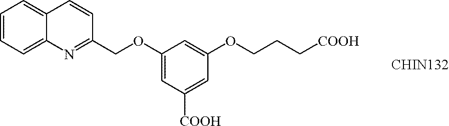 [SMILES: O=C(O)CCCOc1cc(OCc2ccc3ccccc3n2)cc(C(=O)O)c1]

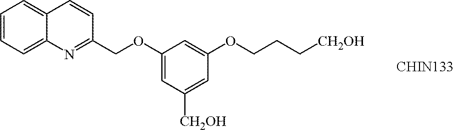 [SMILES: OCCCCOc1cc(CO)cc(OCc2ccc3ccccc3n2)c1]

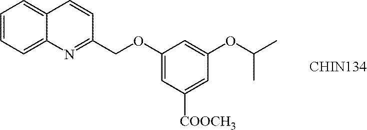 [SMILES: COC(=O)c1cc(OCc2ccc3ccccc3n2)cc(OC(C)C)c1]

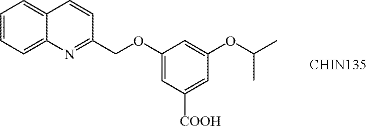 [SMILES: CC(C)Oc1cc(OCc2ccc3ccccc3n2)cc(C(=O)O)c1]

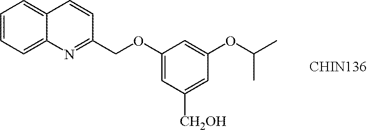 [SMILES: CC(C)Oc1cc(CO)cc(OCc2ccc3ccccc3n2)c1]

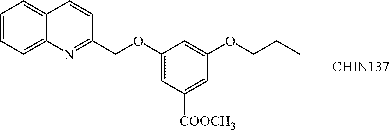 [SMILES: CCCOc1cc(OCc2ccc3ccccc3n2)cc(C(=O)OC)c1]

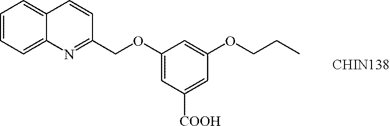 [SMILES: CCCOc1cc(OCc2ccc3ccccc3n2)cc(C(=O)O)c1]

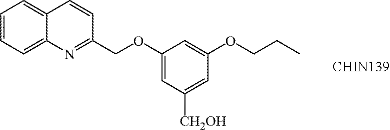 [SMILES: CCCOc1cc(CO)cc(OCc2ccc3ccccc3n2)c1]

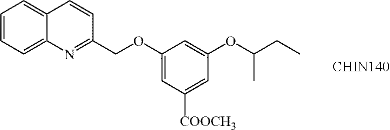 [SMILES: CCC(C)Oc1cc(OCc2ccc3ccccc3n2)cc(C(=O)OC)c1]

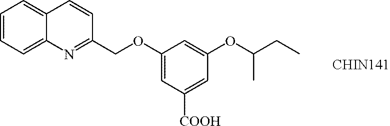 [SMILES: CCC(C)Oc1cc(OCc2ccc3ccccc3n2)cc(C(=O)O)c1]

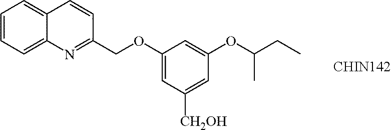 [SMILES: CCC(C)Oc1cc(CO)cc(OCc2ccc3ccccc3n2)c1]

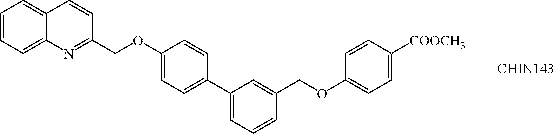 [SMILES: COC(=O)c1ccc(OCc2cccc(-c3ccc(OCc4ccc5ccccc5n4)cc3)c2)cc1]

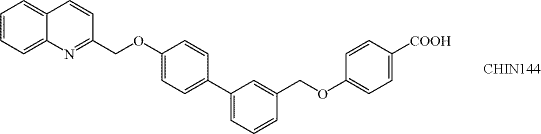 [SMILES: O=C(O)c1ccc(OCc2cccc(-c3ccc(OCc4ccc5ccccc5n4)cc3)c2)cc1]

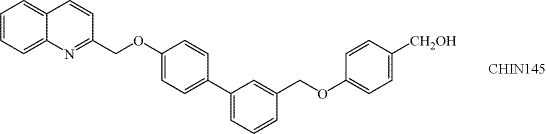 [SMILES: OCc1ccc(OCc2cccc(-c3ccc(OCc4ccc5ccccc5n4)cc3)c2)cc1]

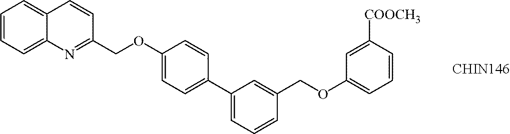 [SMILES: COC(=O)c1cccc(OCc2cccc(-c3ccc(OCc4ccc5ccccc5n4)cc3)c2)c1]

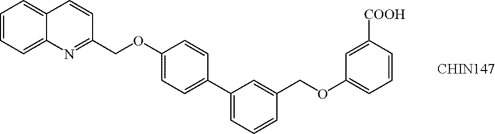 [SMILES: O=C(O)c1cccc(OCc2cccc(-c3ccc(OCc4ccc5ccccc5n4)cc3)c2)c1]

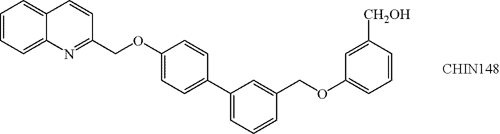 [SMILES: OCc1cccc(OCc2cccc(-c3ccc(OCc4ccc5ccccc5n4)cc3)c2)c1]

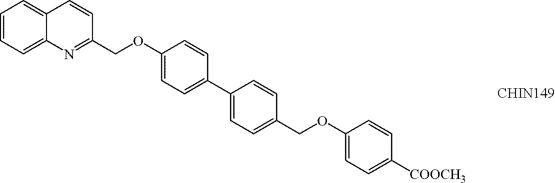 [SMILES: COC(=O)c1ccc(OCc2ccc(-c3ccc(OCc4ccc5ccccc5n4)cc3)cc2)cc1]

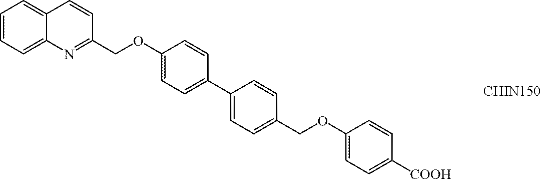 [SMILES: O=C(O)c1ccc(OCc2ccc(-c3ccc(OCc4ccc5ccccc5n4)cc3)cc2)cc1]

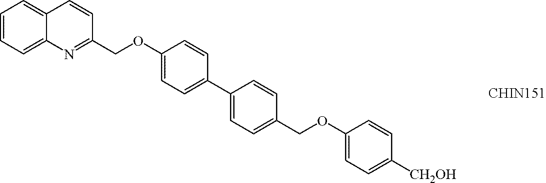 [SMILES: OCc1ccc(OCc2ccc(-c3ccc(OCc4ccc5ccccc5n4)cc3)cc2)cc1]

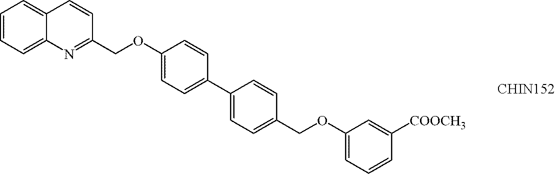 [SMILES: COC(=O)c1cccc(OCc2ccc(-c3ccc(OCc4ccc5ccccc5n4)cc3)cc2)c1]

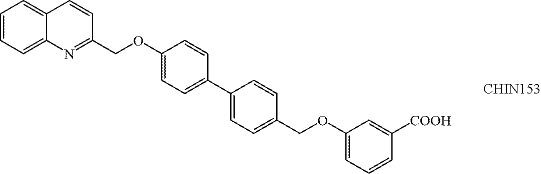 [SMILES: O=C(O)c1cccc(OCc2ccc(-c3ccc(OCc4ccc5ccccc5n4)cc3)cc2)c1]

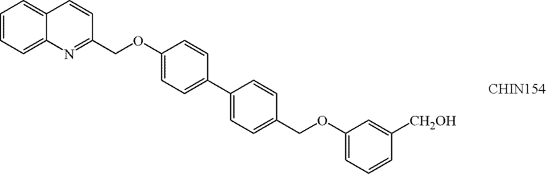 [SMILES: OCc1cccc(OCc2ccc(-c3ccc(OCc4ccc5ccccc5n4)cc3)cc2)c1]

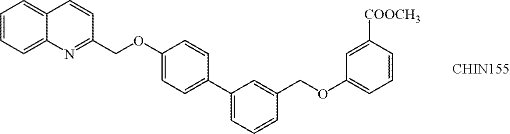 [SMILES: COC(=O)c1cccc(OCc2cccc(-c3ccc(OCc4ccc5ccccc5n4)cc3)c2)c1]

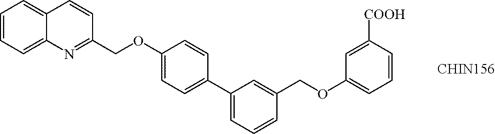 [SMILES: O=C(O)c1cccc(OCc2cccc(-c3ccc(OCc4ccc5ccccc5n4)cc3)c2)c1]

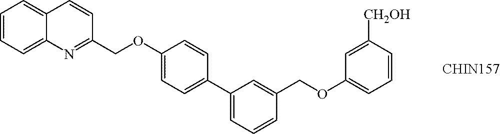 [SMILES: OCc1cccc(OCc2cccc(-c3ccc(OCc4ccc5ccccc5n4)cc3)c2)c1]

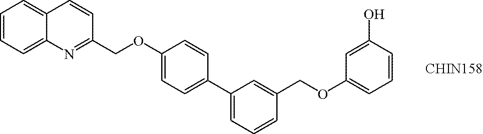 [SMILES: Oc1cccc(OCc2cccc(-c3ccc(OCc4ccc5ccccc5n4)cc3)c2)c1]

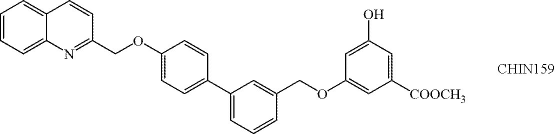 [SMILES: COC(=O)c1cc(O)cc(OCc2cccc(-c3ccc(OCc4ccc5ccccc5n4)cc3)c2)c1]

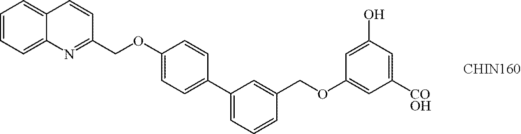 [SMILES: O=C(O)c1cc(O)cc(OCc2cccc(-c3ccc(OCc4ccc5ccccc5n4)cc3)c2)c1]

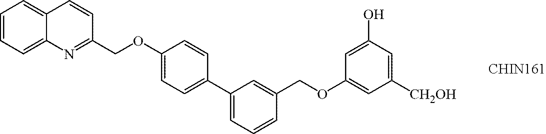 [SMILES: OCc1cc(O)cc(OCc2cccc(-c3ccc(OCc4ccc5ccccc5n4)cc3)c2)c1]

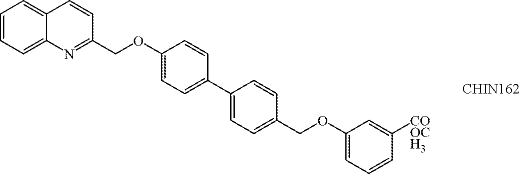 [SMILES: COC(=O)c1cccc(OCc2ccc(-c3ccc(OCc4ccc5ccccc5n4)cc3)cc2)c1]

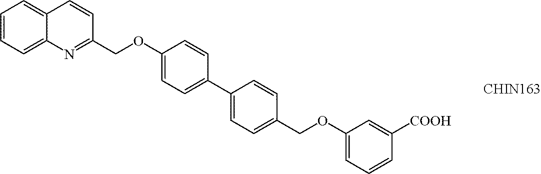 [SMILES: O=C(O)c1cccc(OCc2ccc(-c3ccc(OCc4ccc5ccccc5n4)cc3)cc2)c1]

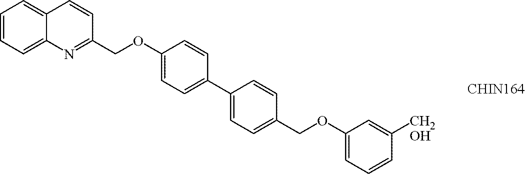 [SMILES: OCc1cccc(OCc2ccc(-c3ccc(OCc4ccc5ccccc5n4)cc3)cc2)c1]

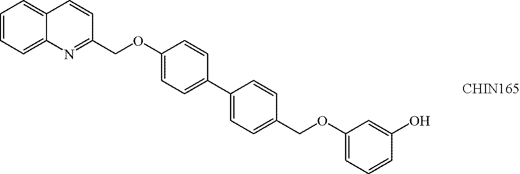 [SMILES: Oc1cccc(OCc2ccc(-c3ccc(OCc4ccc5ccccc5n4)cc3)cc2)c1]

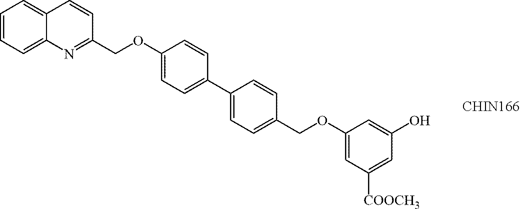 [SMILES: COC(=O)c1cc(O)cc(OCc2ccc(-c3ccc(OCc4ccc5ccccc5n4)cc3)cc2)c1]

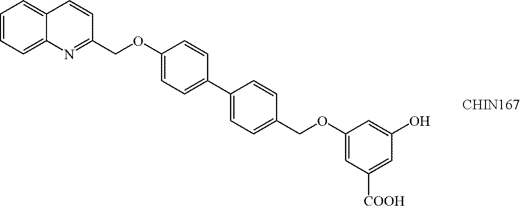 [SMILES: O=C(O)c1cc(O)cc(OCc2ccc(-c3ccc(OCc4ccc5ccccc5n4)cc3)cc2)c1]

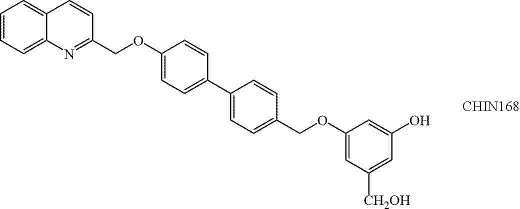 [SMILES: OCc1cc(O)cc(OCc2ccc(-c3ccc(OCc4ccc5ccccc5n4)cc3)cc2)c1]

A second aspect of the present invention relates to a pharmaceutical composition comprising a compound of Formula (Ia) and at least a pharmaceutically acceptable excipient. The compounds of formula (Ia) have formula:

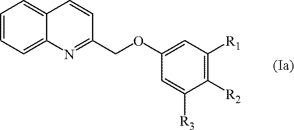

or pharmaceutically acceptable salts or solvates thereof wherein:
R₁ is selected from the group consisting of H, linear or branched C₁₋₆ alkyl optionally substituted with one substituent R₇; linear or branched O-C₃₋₆ alkyl optionally substituted with one substituent R₈;
R₂ is selected from the group consisting of H, C₁₋₆ hydroxyalkyl, linear or branched C₁₋₆ alkyl optionally substituted with one substituent R₇; phenyl optionally substituted with at least one substituent independently selected from the group consisting of H, COOH, COO-C₁₋₆ alkyl, C₁₋₆ hydroxyalkyl and linear or branched C₁₋₆ alkyl optionally substituted with one substituent R₉;
R₃ is selected from the group consisting of H, COOH, COO-C₁₋₆ alkyl, CH₂OH;
provided that:
   when R₂ is H, R₁ or R₃ are not H; or
   when R₁ is linear O-C₃₋₆ alkyl optionally substituted with one substituent R₈, at least one between R₂ and R₃ is not H;
R₄ is selected from the group consisting of H, COOH, COO-C₁₋₆ alkyl, C₁₋₆ hydroxyalkyl;
R₅ is selected from the group consisting of H, COOH, COO-C₁₋₆ alkyl, C₁₋₆ hydroxyalkyl;
R₆ is selected from the group consisting of H, COOH, COO-C₁₋₆ alkyl, C₁₋₆ hydroxyalkyl, OH and linear or branched C₁₋₆ alkoxyl optionally substituted with one substituent R₈;
provided that when one between R₄, R₅ and R₆ is COOH or CH₂OH, at least one of the remaining ones between R₄, R₅ and R₅ is not H,
R₇ is selected from the group consisting of:

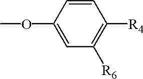

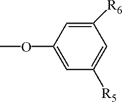

R₈ is selected from the group consisting of OH, COOH, COO-C₁₋₆ alkyl;
R₉ is selected from the group consisting of:

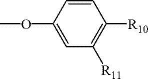

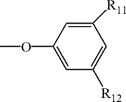

where R₁₀ is selected from the group consisting of H, COOH, COO-C₁₋₆ alkyl, C₁₋₆ hydroxyalkyl;
R₁₁ is selected from the group consisting of H, OH, COOH, COO-C₁₋₆ alkyl, C₁₋₆ hydroxyalkyl;
and R₁₂ is selected from the group consisting of H, COOH, COO-C₁₋₆ alkyl, C₁₋₆ hydroxyalkyl.

In one embodiment R₁ is selected from the group consisting of H, O-i-propyl, O-n-propyl, O-sec-butyl, O-n-pentyl, O-2-methylbutyl, -CH₂-R₇, -O- (CH₂) ₃₋₄-R₈.

In one embodiment, R₂ is a phenyl optionally substituted with at least one substituent independently selected from the group consisting of H, COOH, COO-C₁₋₆ alkyl, C₁₋₆ hydroxyalkyl and linear or branched C₁₋₆ alkyl optionally substituted with one substituent R₉.

In one embodiment, R₂ is selected from the group consisting of H, CH₂OH, phenyl substituted with two substituents independently selected from the group consisting of H, COOH, COO-C₁₋₆ alkyl, C₁₋₆ hydroxyalkyl, -CH₂-R₉.

### In One Embodiment,

R₃ is selected from the group consisting of H, COOH, COOCH₃, CH₂OH;
R₄ is selected from the group consisting of H, COOH, COOCH₃, CH₂OH; and
R₅ is selected from the group consisting of H, COOH, COOCH₃, CH₂OH.

In one embodiment, the compounds of formula (Ia) are selected from the group consisting of:

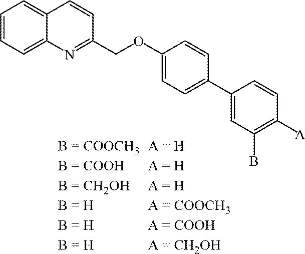

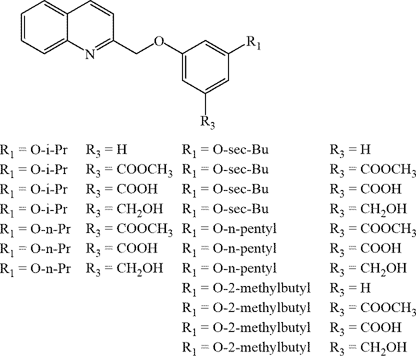

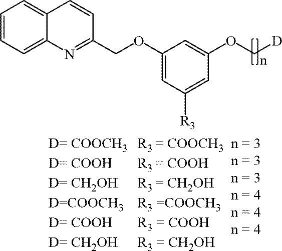

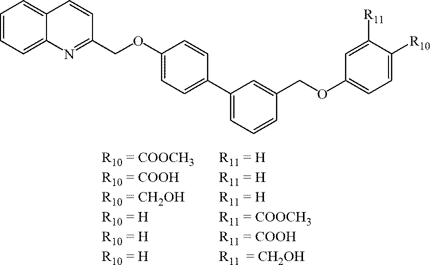

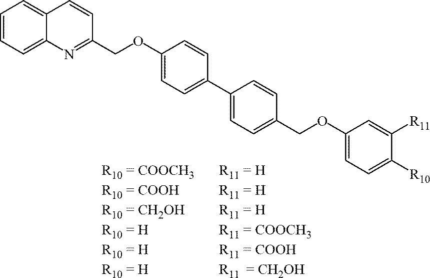

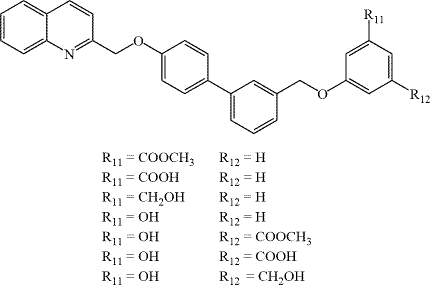

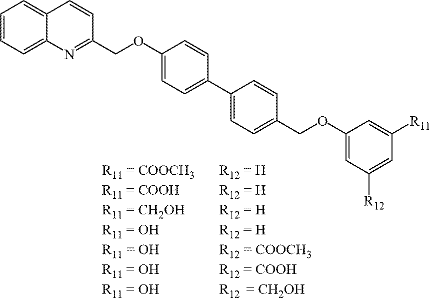

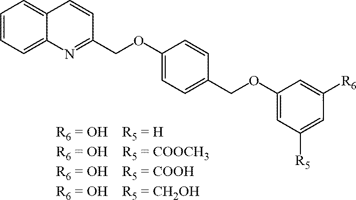

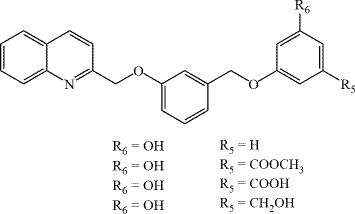

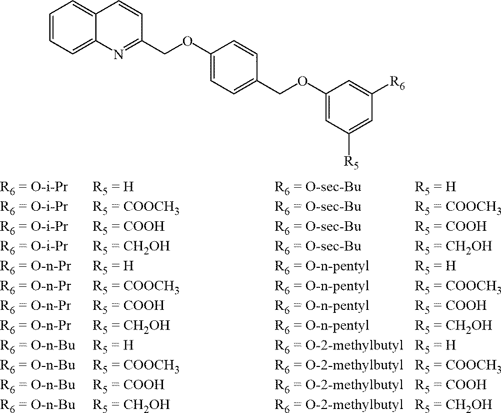

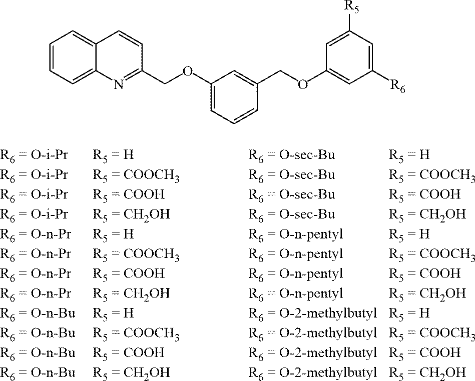

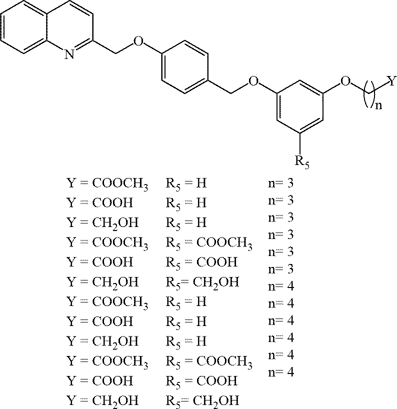

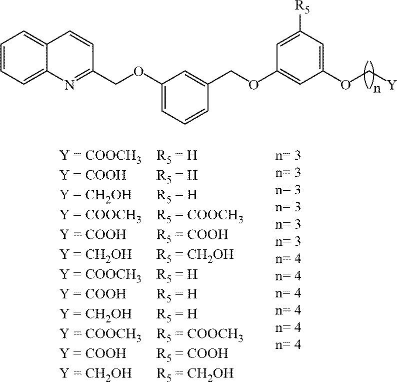

Preferably, the compounds of formula (Ia) are selected from the group consisting of:

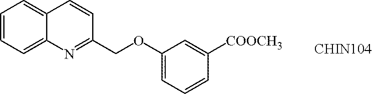 [SMILES: COC(=O)c1cccc(OCc2ccc3ccccc3n2)c1]

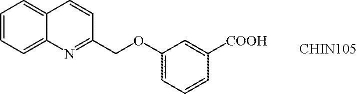 [SMILES: O=C(O)c1cccc(OCc2ccc3ccccc3n2)c1]

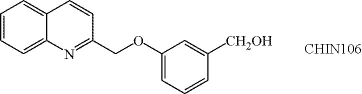 [SMILES: OCc1cccc(OCc2ccc3ccccc3n2)c1]

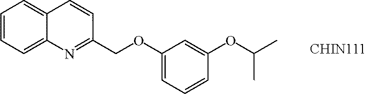 [SMILES: CC(C)Oc1cccc(OCc2ccc3ccccc3n2)c1]

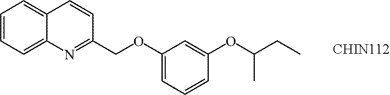 [SMILES: CCC(C)Oc1cccc(OCc2ccc3ccccc3n2)c1]

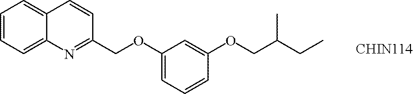 [SMILES: CCC(C)COc1cccc(OCc2ccc3ccccc3n2)c1]

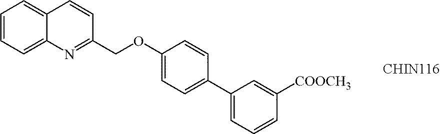 [SMILES: COC(=O)c1cccc(-c2ccc(OCc3ccc4ccccc4n3)cc2)c1]

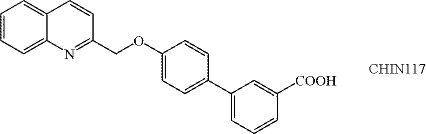 [SMILES: O=C(O)c1cccc(-c2ccc(OCc3ccc4ccccc4n3)cc2)c1]

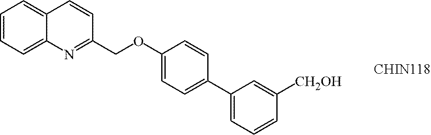 [SMILES: OCc1cccc(-c2ccc(OCc3ccc4ccccc4n3)cc2)c1]

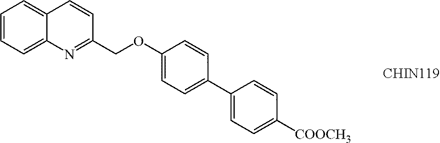 [SMILES: COC(=O)c1ccc(-c2ccc(OCc3ccc4ccccc4n3)cc2)cc1]

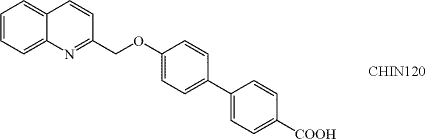 [SMILES: O=C(O)c1ccc(-c2ccc(OCc3ccc4ccccc4n3)cc2)cc1]

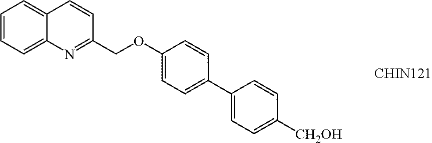 [SMILES: OCc1ccc(-c2ccc(OCc3ccc4ccccc4n3)cc2)cc1]

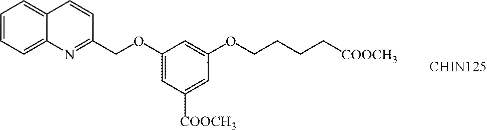 [SMILES: COC(=O)CCCCOc1cc(OCc2ccc3ccccc3n2)cc(C(=O)OC)c1]

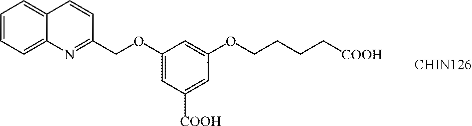 [SMILES: O=C(O)CCCCOc1cc(OCc2ccc3ccccc3n2)cc(C(=O)O)c1]

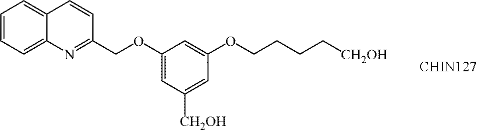 [SMILES: OCCCCCOc1cc(CO)cc(OCc2ccc3ccccc3n2)c1]

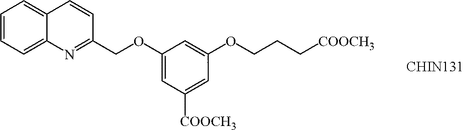 [SMILES: COC(=O)CCCOc1cc(OCc2ccc3ccccc3n2)cc(C(=O)OC)c1]

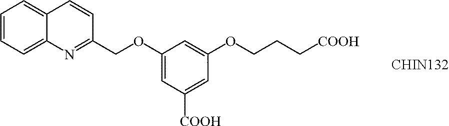 [SMILES: O=C(O)CCCOc1cc(OCc2ccc3ccccc3n2)cc(C(=O)O)c1]

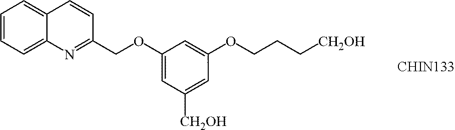 [SMILES: OCCCCOc1cc(CO)cc(OCc2ccc3ccccc3n2)c1]

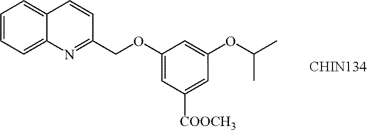 [SMILES: COC(=O)c1cc(OCc2ccc3ccccc3n2)cc(OC(C)C)c1]

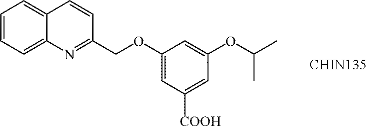 [SMILES: CC(C)Oc1cc(OCc2ccc3ccccc3n2)cc(C(=O)O)c1]

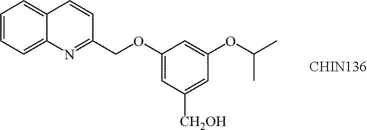 [SMILES: CC(C)Oc1cc(CO)cc(OCc2ccc3ccccc3n2)c1]

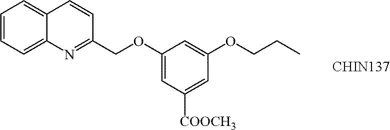 [SMILES: CCCOc1cc(OCc2ccc3ccccc3n2)cc(C(=O)OC)c1]

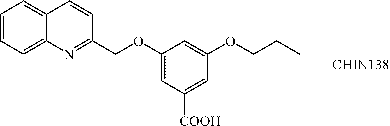 [SMILES: CCCOc1cc(OCc2ccc3ccccc3n2)cc(C(=O)O)c1]

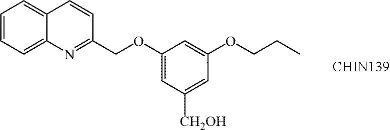 [SMILES: CCCOc1cc(CO)cc(OCc2ccc3ccccc3n2)c1]

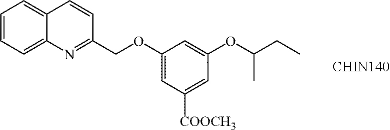 [SMILES: CCC(C)Oc1cc(OCc2ccc3ccccc3n2)cc(C(=O)OC)c1]

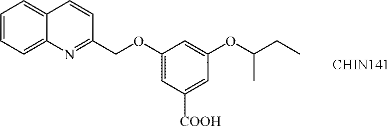 [SMILES: CCC(C)Oc1cc(OCc2ccc3ccccc3n2)cc(C(=O)O)c1]

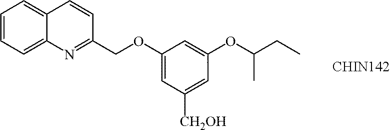 [SMILES: CCC(C)Oc1cc(CO)cc(OCc2ccc3ccccc3n2)c1]

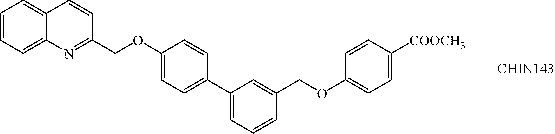 [SMILES: COC(=O)c1ccc(OCc2cccc(-c3ccc(OCc4ccc5ccccc5n4)cc3)c2)cc1]

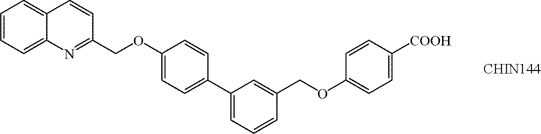 [SMILES: O=C(O)c1ccc(OCc2cccc(-c3ccc(OCc4ccc5ccccc5n4)cc3)c2)cc1]

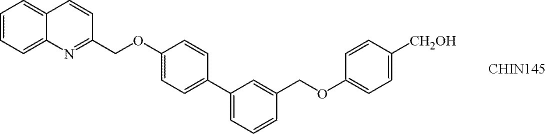 [SMILES: OCc1ccc(OCc2cccc(-c3ccc(OCc4ccc5ccccc5n4)cc3)c2)cc1]

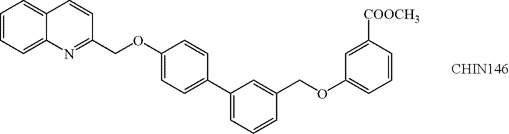 [SMILES: COC(=O)c1cccc(OCc2cccc(-c3ccc(OCc4ccc5ccccc5n4)cc3)c2)c1]

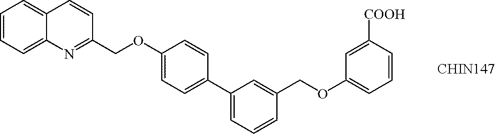 [SMILES: O=C(O)c1cccc(OCc2cccc(-c3ccc(OCc4ccc5ccccc5n4)cc3)c2)c1]

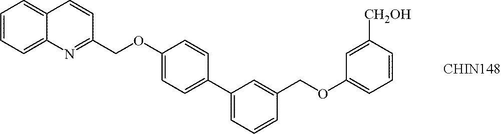 [SMILES: OCc1cccc(OCc2cccc(-c3ccc(OCc4ccc5ccccc5n4)cc3)c2)c1]

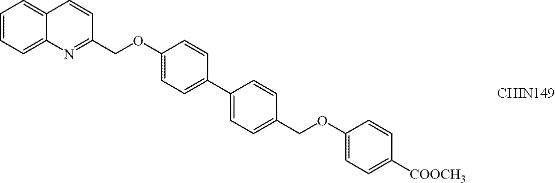 [SMILES: COC(=O)c1ccc(OCc2ccc(-c3ccc(OCc4ccc5ccccc5n4)cc3)cc2)cc1]

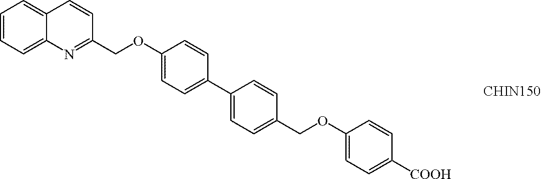 [SMILES: O=C(O)c1ccc(OCc2ccc(-c3ccc(OCc4ccc5ccccc5n4)cc3)cc2)cc1]

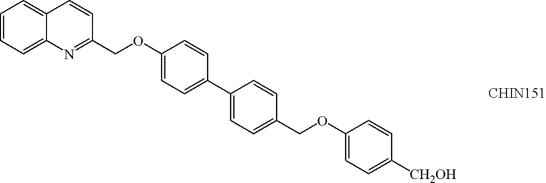 [SMILES: OCc1ccc(OCc2ccc(-c3ccc(OCc4ccc5ccccc5n4)cc3)cc2)cc1]

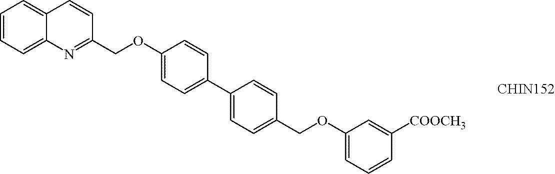 [SMILES: COC(=O)c1cccc(OCc2ccc(-c3ccc(OCc4ccc5ccccc5n4)cc3)cc2)c1]

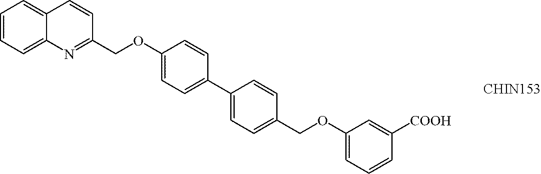 [SMILES: O=C(O)c1cccc(OCc2ccc(-c3ccc(OCc4ccc5ccccc5n4)cc3)cc2)c1]

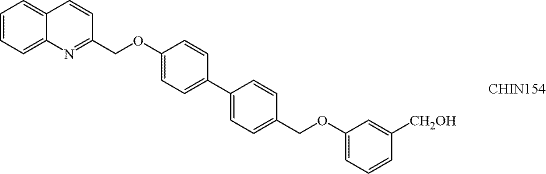 [SMILES: OCc1cccc(OCc2ccc(-c3ccc(OCc4ccc5ccccc5n4)cc3)cc2)c1]

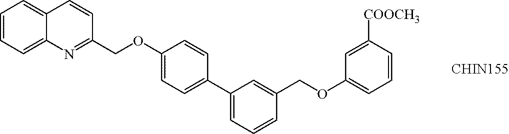 [SMILES: COC(=O)c1cccc(OCc2cccc(-c3ccc(OCc4ccc5ccccc5n4)cc3)c2)c1]

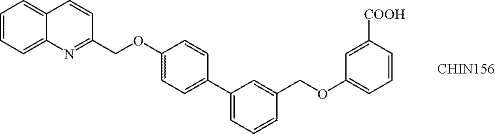 [SMILES: O=C(O)c1cccc(OCc2cccc(-c3ccc(OCc4ccc5ccccc5n4)cc3)c2)c1]

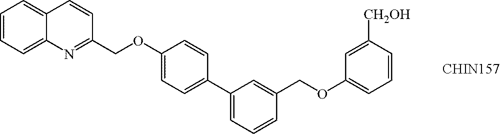 [SMILES: OCc1cccc(OCc2cccc(-c3ccc(OCc4ccc5ccccc5n4)cc3)c2)c1]

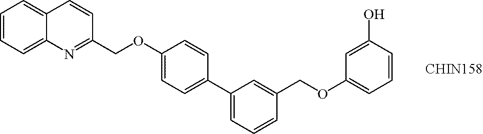 [SMILES: Oc1cccc(OCc2cccc(-c3ccc(OCc4ccc5ccccc5n4)cc3)c2)c1]

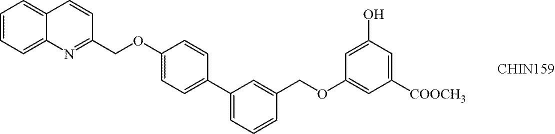 [SMILES: COC(=O)c1cc(O)cc(OCc2cccc(-c3ccc(OCc4ccc5ccccc5n4)cc3)c2)c1]

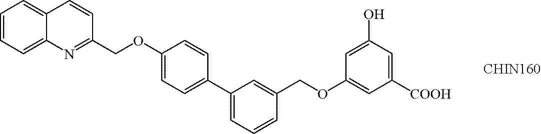 [SMILES: O=C(O)c1cc(O)cc(OCc2cccc(-c3ccc(OCc4ccc5ccccc5n4)cc3)c2)c1]

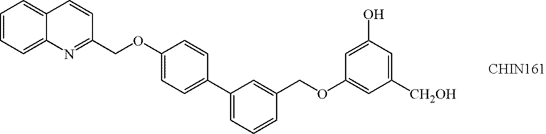 [SMILES: OCc1cc(O)cc(OCc2cccc(-c3ccc(OCc4ccc5ccccc5n4)cc3)c2)c1]

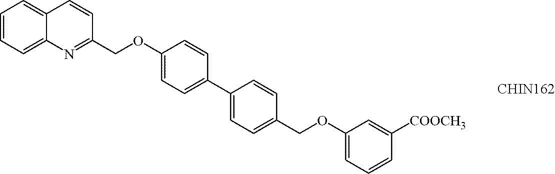 [SMILES: COC(=O)c1cccc(OCc2ccc(-c3ccc(OCc4ccc5ccccc5n4)cc3)cc2)c1]

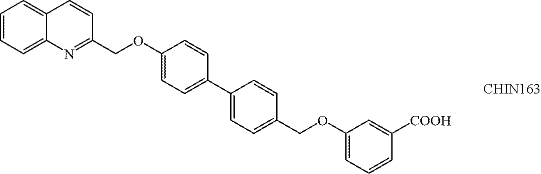 [SMILES: O=C(O)c1cccc(OCc2ccc(-c3ccc(OCc4ccc5ccccc5n4)cc3)cc2)c1]

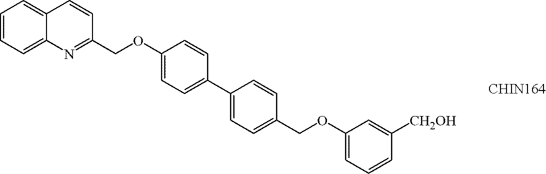 [SMILES: OCc1cccc(OCc2ccc(-c3ccc(OCc4ccc5ccccc5n4)cc3)cc2)c1]

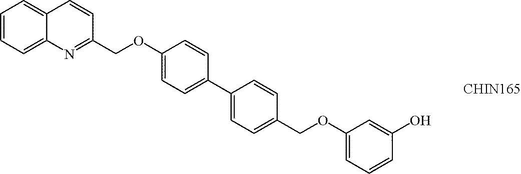 [SMILES: Oc1cccc(OCc2ccc(-c3ccc(OCc4ccc5ccccc5n4)cc3)cc2)c1]

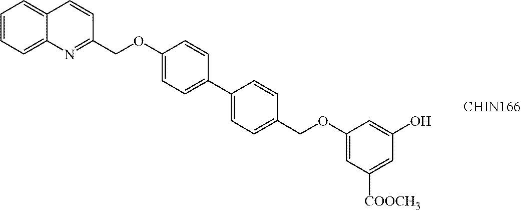 [SMILES: COC(=O)c1cc(O)cc(OCc2ccc(-c3ccc(OCc4ccc5ccccc5n4)cc3)cc2)c1]

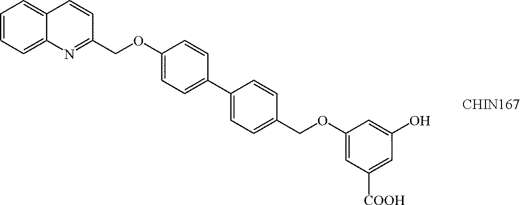 [SMILES: O=C(O)c1cc(O)cc(OCc2ccc(-c3ccc(OCc4ccc5ccccc5n4)cc3)cc2)c1]

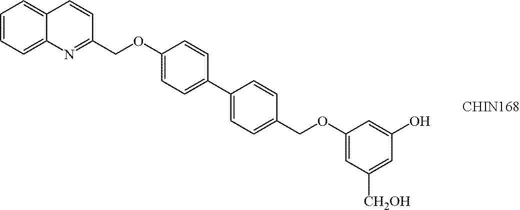 [SMILES: OCc1cc(O)cc(OCc2ccc(-c3ccc(OCc4ccc5ccccc5n4)cc3)cc2)c1]

A person skilled in the art is aware of a whole variety of such excipient compounds suitable for formulating a pharmaceutical composition.

The compounds of formula (Ia), together with a conventionally employed excipient may be included in pharmaceutical compositions and dosage units thereof and in such form may be used as solids, such as tablets or filled capsules, or liquids such as solutions, suspensions, emulsions, elixirs or capsules filled with the same, all for oral use or as sterile injectable solutions for parenteral administration (including subcutaneous and intravenous use).

Such pharmaceutical compositions and the unit dosage forms thereof may comprise ingredients in conventional percentages, with or without additional compounds or active ingredients, and such unit dosage forms may comprise any suitable effective amount of the active ingredient commensurate with the intended daily dosage range to be employed.

The pharmaceutical compositions containing a compound of the present invention can be prepared in a manner well known in the pharmaceutical art and comprise at least one active compound. Generally, the compounds of the present invention are administered in a pharmaceutically effective amount. The amount of compound actually administered will typically be determined by a physician, taking into account relevant circumstances, including the condition to be treated, the route of administration chosen, the actual compound administered, the age, weight and response of the individual patient, the severity of the patient’s symptoms, and the like.

The pharmaceutical compositions of the present invention can be administered by numerous routes including oral, rectal, subcutaneous, intravenous, intramuscular, intranasal and pulmonary routes. The compositions for oral administration can take the form of liquid solutions or suspensions in bulk or in bulk powders. More commonly, however, the compositions are presented in unit dosage forms to facilitate the precise dosing. The expression “unit dosage forms” refers to physically distinct units suitable as unit dosages for human and other mammalian subjects, each unit containing a predetermined amount of active material calculated to produce the desired therapeutic effect, in association with an acceptable pharmaceutical excipient. Typical unit dosage forms include pre-filled, pre-dosed ampoules or syringes of the liquid compositions or pills, tablets, capsules or similar in the case of solid compositions.

The liquid forms suitable for oral administration may include a suitable aqueous or non-aqueous vehicle with buffering agents, suspending and dispersing agents, dyes, flavours and the like. The solid forms may include, for example, any of the following ingredients, or compounds of similar nature: a binder such as microcrystalline cellulose, tragacanth gum or gelatin; an excipient such as starch or lactose; a disintegrating agent such as alginic acid, Primogel or corn starch; a lubricant such as magnesium stearate; a flow agent such as colloidal silicon dioxide; a sweetening agent such as sucrose, lactose or saccharin; or a flavouring agent such as peppermint, methyl salicylate or orange flavouring.

The injectable compositions are typically based on sterile injectable solution or phosphate buffered solution or other injectable vehicles known in the art.

The pharmaceutical compositions may be in the form of tablets, pills, capsules, solutions, suspensions, emulsions, powders, suppositories and as sustained release formulations.

If desired, tablets may be coated using standard aqueous or non-aqueous techniques. In certain embodiments, such compositions and preparations may contain at least 0.1 percent of active compound. The percentage of active compound in these compositions can be varied, of course, and can suitably be between about 1 percent and about 60 percent of the unit weight. The amount of active compound in such therapeutically useful compositions is such that the therapeutically active dosage will be obtained. The active compound can also be administered intranasally as, for example, liquid drops or sprays.

The tablets, pills, capsules, and the like may also contain a binder such as tragacanth gum, acacia, corn starch, or jelly; excipients such as dicalcium phosphate; a disintegrating agent such as corn starch, potato starch, alginic acid; a lubricant such as magnesium stearate; and a sweetening agent such as sucrose, lactose, or saccharin. When a dosage unit form is a capsule, it may contain, in addition to the materials of the above type, a liquid carrier such as a fatty oil. Various other materials may be present as coatings or to modify the physical form of the dosing unit. For example, the tablets can be coated with shellac, sugar, or both. A syrup or elixir may contain, in addition to the active ingredient, sucrose as a sweetener, methyl and propyl parabens as preservatives, a dye and a flavouring agent such as cherry or orange flavour. To avoid breakage during the transit through the upper part of the gastrointestinal tract, the composition is an enteric-coated formulation.

The compositions for pulmonary administration include, but are not limited to, dry powder compositions consisting of powder of a compound of formula (Ia) and the powder of a suitable vehicle and/or lubricant. The compositions for pulmonary administration may be inhaled by any suitable dry powder inhaler device known to the person skilled in the art.

The administration of the compositions is performed according to a protocol and at a dosage sufficient to reduce inflammation and pain in the subject. In some embodiments, in the pharmaceutical compositions of the present invention the active ingredient or the active ingredients are generally formulated in dosage units. The dosage unit may contain 0.1 to 1000 mg of a formula compound (Ia) per dosage unit for the daily administration.

In some embodiments, the effective amounts for a specific formulation will depend on the severity of the disease, disorder or condition prior to therapy, the health status of the individual and the response to the drug. In some embodiments the dose is in the range from 0.001% by weight to about 60% by weight of the formulation.

When used in combination with one or more of the other active ingredients, the compound of the present invention and the other active ingredient may be used in lower doses than when each is used individually.

As regards the formulations relating to any variety of routes of administration, methods and formulations for drug administration are described in Remington’s Pharmaceutical Sciences, 17^{th} Edition, Gennaro et al. Ed., Mack Publishing Co., 1985 and Remington’s Pharmaceutical Sciences, Gennaro AR ed. 20^{th} Edition, 2000, Williams & Wilkins PA, USA and Remington: The Science and Practice of Pharmacy, 21^{st} Edition, Lippincott Williams & Wilkins Ed., 2005; and in Loyd V. Allen e Howard C. Ansel, Ansel’s Pharmaceutical Dosage Forms and Drug Delivery Systems, 10^{th} Edition, Lippincott Williams & Wilkins Ed., 2014.

The components described above for orally administered or injectable compositions are only representative.

The compounds of the present invention may also be administered in sustained release forms or by sustained release drug delivery systems.

A third aspect of the present invention relates to the compounds of formula (Ia) as described above for use as a medicament.

A compound of Formula (Ia), as shown above, may be used in the prevention and/or treatment of a disorder selected from the group consisting of gastrointestinal disorders, liver disorders, cardiovascular disorders, metabolic disorders, infectious diseases, cancer, renal disorders, inflammatory disorders and neurological disorders.

In one embodiment, liver disorders include primary biliary cirrhosis (PBC), cerebrotendinous xanthomatosis (CTX), primary sclerosing cholangitis (PSC), drug-induced cholestasis, intrahepatic cholestasis of pregnancy, cholestasis associated with parenteral nutrition, cholestasis associated with bacterial overgrowth and sepsis, autoimmune hepatitis, chronic viral hepatitis, alcoholic liver disease, non-alcoholic fatty liver disease (NAFLD), non-alcoholic steatohepatitis (NASH), liver transplantation-associated host disease, living donor transplantation, liver regeneration, congenital liver fibrosis, granulomatous liver disease, intra- or extrahepatic malignancy, Wilson’s disease, haemochromatosis, and alpha-1-antitrypsin deficiency.

In one embodiment, gastrointestinal disorders include inflammatory bowel disease (IBD) (including Crohn’s disease, ulcerative colitis and indeterminate colitis), irritable bowel syndrome (IBS), bacterial overgrowth, acute and chronic pancreatitis, malabsorption, post-radiation colitis, and microscopic colitis.

In one embodiment, renal disorders include diabetic nephropathy, hypertensive nephropathy, chronic glomerulonephritis including chronic transplant glomerulonephritis, chronic tubulointerstitial disease and vascular disorders of the kidney.

In one embodiment, the cardiovascular disease is selected from the group consisting of atherosclerosis, dyslipidaemia, hypercholesterolemia, hypertriglyceridemia, hypertension also known as high blood pressure, inflammatory heart disease including myocarditis and endocarditis, ischemic heart disease, stable angina, unstable angina, myocardial infarction, cerebrovascular disease including ischaemic stroke, pulmonary heart disease including pulmonary hypertension, peripheral artery disease (PAD), also known as peripheral vascular disease (PVD), peripheral artery occlusive disease and peripheral obliterative arteriopathy.

In one embodiment, the metabolic disease is selected from the group consisting of insulin resistance, metabolic syndrome, type I and type II diabetes, hypoglycaemia, and adrenal cortex disorders including adrenal cortex insufficiency.

In one embodiment, the metabolic disorder is selected from the group consisting of obesity and conditions associated with bariatric surgery.

In one embodiment, cancer is selected from the group comprising liver cancer, bile duct cancers, pancreatic cancer, gastric cancer, colorectal cancer, breast cancer, ovarian cancer and pathology associated with resistance to chemotherapy.

In one embodiment, the infectious disease is selected from the group of acquired immunodeficiency syndrome (AIDS) and related disorders, B virus and C virus infection.

In one embodiment, the inflammatory disorder is selected from the group of rheumatoid arthritis, fibromyalgia, Sjögren’s syndrome, scleroderma, Behcet’s syndrome, vasculitis and systemic lupus erythematosus.

According to a further aspect of the invention, compounds of formula (Ia) are provided for use as selective agonists of GPBAR1. In particular, CHIN114.

According to a further aspect of the invention, compounds of formula (Ia) are provided for use as dual CysLT1R/FXR modulators. A favourite example of such compounds of formula I is CHIN104.

According to a further aspect of the invention, compounds of formula (Ia) are provided for use as dual CysLT1R/GPBAR1 modulators. Preferred examples of such compounds of formula (Ia) are CHIN105, CHIN106, and CHIN117.

Further characteristics of the present invention will become apparent from the following description of some merely illustrative and non-limiting examples.

The following abbreviations are used in the attached examples: methanol (MeOH), sodium bicarbonate (NaHCO₃), ethyl acetate (EtOAc), dichloromethane(DCM), sodium sulfate (Na₂SO₄), dimethylformamide (DMF), diisobutylaluminium hydride (DIBAL-H), triphenylphosphine (PPh₃), diisopropylazodicarboxylate (DIAD), hydrochloric acid (HCl), triethylamine (TEA), trifluoroacetic acid (TFA), sodium hydroxide (NaOH), tetrahydrofuran (THF), water (H₂O), chloroform deuterate (CDCl₃), methanol deuterate (CD₃OD), time (h), room temperature (rt), retention time (t_{R}).

### EXAMPLES

### Example 1. Synthesis of CHIN104-106

The alcohol 1 was synthesised from Methyl quinoline-2-carboxylate by reduction with diisobutylaluminium hydride (DIBAL-H). The alcohol 1 thus obtained is a substrate of the Mitsunobu reaction with methyl 3-hydroxybenzoate (diagram 1) to synthesise CHIN104 in high yields.

The basic hydrolysis or the reduction with DIBAL-H of the methyl ester provided, starting from CHIN104, the carboxylic acids CHIN105 and the alcohol CHIN106, respectively.

Diagram 1

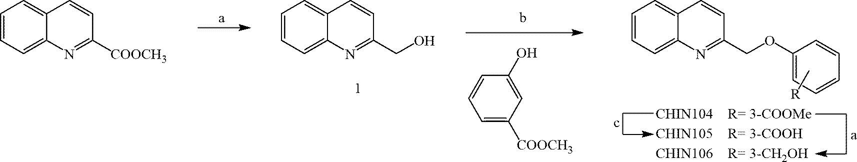

Reagents and conditions a) DIBAL-H, dry THF, 0° C.; b) PPh₃, DIAD, dry THF, 0° C.; c) NaOH, MeOH: H₂O 1:1 v/v.

### General Processes

Reaction a). Reduction with DIBAL-H. A solution of DIBAL-H (2.0 equiv., 1.0 M in THF) is added drop by drop to a solution of quinoline methylester or alternatively of CHIN104 in anhydrous THF (25 mL) at 0° C. The resulting mixture is stirred for 4 h-8 h at 0° C. A saturated aqueous solution of Rochelle salt (sodium potassium tartrate) is added to the reaction mixture and subsequently diluted with DCM. Quenching lasts 2 hours under stirring. The aqueous phase is extracted with DCM (3 x 50 mL) and the pooled organic phases are washed with water, anhydrified with Na₂SO₄ and concentrated under vacuum on the rotavapor to obtain a crude residue which is purified by chromatographic column or HPLC.

Step b) Mitsunobu reaction. Diisopropylazodicarboxylate (DIAD, 3.5 equiv.) is added drop by drop to a solution of triphenylphosphine (PPh₃, 3.5 equiv.) in dry THF at 0° C. After 10 minutes, a solution of alcohol 1 dissolved in dry THF is added. After a further 10 minutes, a solution of methyl 3-hydroxybenzoate solubilised in dry THF is added. After about 12 hours, water is added and the reaction mixture is dried to remove the THF. The dry residue is extracted with EtOAc (3 x 50 mL), and the pooled organic phases are washed with an aqueous solution of 2.5 M KOH and water, anhydrified and dried under vacuum on the rotavapor. The purification on a chromatographic column and silica gel provided CHIN104.

Step c) Basic hydrolysis. A small aliquot of the CHIN104 ester is dissolved in a solution of MeOH: H₂O 1:1 v/v (30 mL) and treated in basic environment for NaOH (5.0 equiv.). The reaction mixture is stirred for 8 h under reflux at a temperature of about 150° C. The resulting solution is quenched by treatment with 6 M HCl and then extracted with EtOAc (3 x 50 mL). The pooled organic phases are washed with water, treated with anhydrous Na₂SO₄ and then dried on the rotavapor to give CHIN105 as a crude residue, which is subject to further purification.

### Example 1A. Synthesis of Methyl 3-(Quinolin-2-ylmethoxy)Benzoate (CHIN104)

The purification was obtained by means of silica gel using hexane as the eluent mixture: EtOAc 9:1 v/v and 0.1% of TEA provided CHIN104 (78%). An analytical sample was obtained by HPLC separation on a Nucleodur 100-5 C18 column (5 µm; 10 mm i.d. x 250 mm), and with MeOH/H₂O 82:18 v/v as eluent (flow 3 mL/min, t_{R} = 14.8 min).

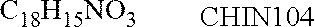

¹H NMR (CDCl₃, 400 MHz): δ 8.22 (1H, d, J = 8.4 Hz), 8.10 (1H, d, J = 8.0 Hz), 7.85 (1H, d, J = 8.0 Hz), 7.74 (2H, ovl), 7.68 (2H, ovl), 7.57 (1H, t, J = 8.0 Hz), 7.37 (1H, t, J = 7.7 Hz), 7.24 (1H, d, J = 7.7 Hz), 5.44 (2H, s), 3.91 (3H, s).

¹³C NMR (CDCl₃, 100 MHz) δ 169.6, 158.4, 157.3, 147.5, 137.1, 131.6, 129.8, 129.5, 128.9, 127.7, 127.6, 126.6, 122.4, 119.7, 119.0, 115.6, 71.4, 52.1.

### Example 2A. Synthesis of 3-(quinolin-2-ylmethoxy)benzoic Acid (CHIN105)

The purification of CHIN105 (68%) was performed on a silica chromatographic column, using DCM as eluent: MeOH 99:1 v/v. An analytical sample was purified by HPLC, using a Nucleodur 100-5 column (5 µm; 10 mm i.d. × 250 mm), and an eluent mixture hexane/EtOAc 40:60 v/v (flow 3 mL/min, t_{R} = 6.9 min).

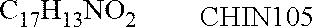

¹H NMR (CD₃OD, 400 MHz) : δ 8.40 (1H, d, J = 8.5 Hz), 8.06 (1H, d, J = 8.3 Hz), 7.95 (1H, d, J = 8.3 Hz), 7.80 (1H, t, J = 8.3 Hz), 7.74 (1H, d, J = 8.5 Hz), 7.70 (1H, s), 7.64 (1H, t, J = 8.3 Hz), 7.62 (1H, d, ovl), 7.41 (1H, t), 7.30 (1H, dd, J = 1.5, 8.0 Hz), 5.42 (2H, s).

¹³C NMR (CD₃OD, 100 MHz) δ 169.4, 159.9, 158.8, 148.4, 139.1, 133.5, 131.3, 130.7, 129.2, 129.1, 129.0, 128.0, 123.7, 120.8, 120.7, 116.6, 71.9.

### Example 3A. Synthesis of (3-(quinolin-2-ylmethoxy)phenyl)methanol (CHIN106).

The purification by silica column using DCM as eluent: MeOH 99:1 v/v provided CHIN106 (60%). An analytical sample was obtained by HPLC separation on a Nucleodur 100-5 C18 column (5 µm; 10 mm i.d. x 250 mm), with MeOH/H₂O 75:15 as eluent (flow 3 mL/min, t_{R} = 9.3 min).

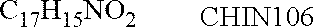

¹H NMR (CDCl₃, 400 MHz): δ 8.20 (1H, d, J = 8.4 Hz), 8.10 (1H, d, J = 7.4 Hz), 7.84 (1H, d, J = 7.4 Hz), 7.75 (1H, t, J = 7.4 Hz), 7.68 (1H, d, J = 8.4 Hz), 7.56 (1H, t, J = 7.4 Hz), 7.28 (1H, dd, J = 7.3, 8.0 Hz), 7.08 (1H, s), 7.0 (1H, d, J = 8.4 Hz), 6.95 (1H, d, J = 7.3 Hz), 5.40 (2H, s), 4.68 (2H, s). ¹³C NMR (CDCl₃, 100 MHz) δ 158.6, 157.8, 147.4, 142.8, 137.1, 129.8, 129.6, 128.7, 127.7, 127.6, 126.5, 119.6, 119.1, 113.9, 113.4, 71.1, 64.9.

### Example 2 Synthesis of CHIN107, CHIN108 and CHIN109

For the synthesis of the compounds CHIN107-CHIN109, the first step involves the monoprotection with TBS of methyl 3,5-dihydroxybenzoate, which must bind to quinoline. Once the monoprotected derivative is obtained, it will be bound to quinoline through a Williamson reaction between the phenol and the mesylated alcohol derivative 1. The final step is the deprotection of TBS with tetrabutylammonium fluoride (TBAF) in order to obtain CHIN107. On two aliquots of the ester, the basic hydrolysis and the reduction with DIBAL-H are carried out to obtain CHIN108 and CHIN109.

Diagram 2.

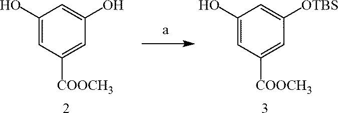

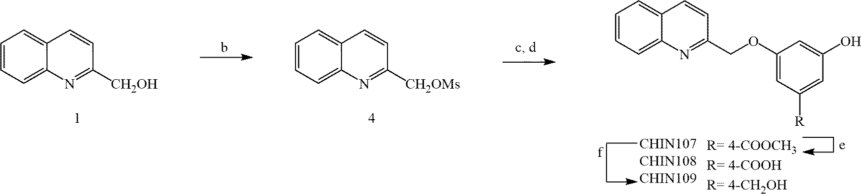

Reagents and conditions: a) TBS-Cl, imidazole, dry DMF, 45% yield; b) Mesylchloride, TEA, ether, -20° C., quantitative yield c) phenol (compound 3), K₂CO₃, anhydrous DMF, 100° C.; d) tetrabutylammonium fluoride (TBAF) 1.0 M in dry THF, over-night; e) NaOH tablets in excess, MeOH:H₂O 1:1 v/v, over-night, to be refluxed; f) DIBAL-H, dry THF, 0° C.

### General Processes

Reaction a). Protection with TBSCl. Imidazole (1.5 equiv.) and tert-butyl dimethylsilyl chloride (1.2 equiv.) are added to a solution of the compound 2 in dry DMF. After one hour the DMF is removed, extracted with EtOAc/NH₄Cl three times and the pooled organic phases are washed with H₂O. The organic phase is anhydrified (Na₂SO₄), filtered, concentrated in the rotavapor, obtaining the compound 3 in the crude state. The purification on silica gel using a hexane/EtOAc 9:1 mixture as eluent provided the compound 3 with 45% yield.

Reaction b). Mesylation of alcohol 1. The compound 1 is solubilised in dry ether and triethylamine (6 equiv.) and methanesulphonyl chloride (5 equiv.) are added to the solution at -20° C. After about 1 h, the solution is washed with a saturated aqueous solution of NaHCO₃ and the pooled organic phases are extracted for once with water. The organic phase is anhydrified (Na₂SO₄), filtered, concentrated in the rotavapor, obtaining the compound 4 in the crude state with a quantitative yield.

Reaction c). Williamson’s reaction. Potassium carbonate (2.5 equiv.) is added to the phenol solution (compound 3) in DMF and left for 15 minutes. The mesylate derivative (1.2 equiv., compound 4) dissolved in dry DMF is added and the solution is placed at 100° C. for about 12 h. The DMF is cooled and removed on the rotavapor and the solid residue is extracted with water and ethyl acetate (3 × 50 mL). The pooled organic phases are anhydrified with Na₂SO₄, filtered, and then concentrated on the rotavapor, obtaining a crude reaction product, which will be directly submitted to the next reaction.

Reaction d) Deprotection from TBS. The crude product from the previous reaction is dissolved in dry THF at room temperature and 1.0 M TBAF tetra-N-butylammonium fluoride solution in THF (0.63 mL, 5 equiv.) is added to the solution. The reaction is finished after 8 hours, and is treated by adding AcOEt and extracting with H₂O. The combined organic phases are anhydrified with Na₂SO₄, filtered, concentrated on the rotavapor, obtaining the compound CHIN107 in its crude state.

Reaction e) Alkaline hydrolysis. The same synthesis and work-up process is carried out as in example 1 step c).

Reaction f) Ester reduction with DIBAL-H. The same synthesis and work-up process as in example 1 step a) is carried out.

Example 2A. Synthesis of methyl 3-hydroxy-5-(quinolin-2-ylmethoxy)benzoate (CHIN107).

The purification is performed on a silica gel packed column, using a DCM/MeOH 998:2 mixture as eluent and obtaining the compound CHIN107 with 85% yield.

An analytical sample is separated in HPLC on a direct-phase semi-preparative Nucleodur 100-5 column (5 µm; 10 mm i.d. x 250 mm) and using hexane/AcOEt 7:3 v/v as eluent mixture (flow at 3 mL/min, t_{R} = 23.70 min).

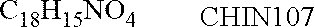

¹H NMR (400 MHz, CDCl₃): δ 8.17 (1H, d, J = 8.5 Hz), 8.00 (1H, d, J = 8.0 Hz), 7.79 (1H, d, J = 8.0 Hz), 7.68 (1H, t, J = 8.0 Hz), 7.62 (1H, t, J = 8.5 Hz), 7.54 (1H, t, J = 8.0 Hz), 7.28 (1H, s), 7.21 (1H, s), 6.76 (1H, s), 5.38 (2H, s), 3.89 (3H, s).

¹³C NMR (100 MHz, CDC1₃): δ 167.2, 159.1, 157.9, 157.3, 146.7, 137.8, 131.8, 130.2, 127.8, 127.7, 127.6, 126.8, 119.2, 109.8, 107.8, 106.8, 70.4, 52.1.

### Example 2B. Synthesis of 3-hydroxy-5-(quinolin-2-ylmethoxy)benzoic Acid (CHIN108).

The purification is performed on a silica gel packed column, using a DCM/MeOH 95:5 mixture as eluent and obtaining the compound CHIN108 with a quantitative yield. A pure analytical sample was obtained by separating the mixture in HPLC on a Phenomenex pentafluorophenyl C18 reverse phase column and using MeOH/H₂O 55:45 v/v and 0.1% of TFA as eluent mixture (flow at 1 mL/min, t_{R} = 9.25 min) .

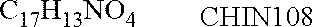

¹H NMR (400 MHz, CD₃OD): δ 8.39 (1H, d, J = 8.4 Hz), 8.05 (1H, d, J = 8.0 Hz), 7.96 (1H, d, J = 8.0 Hz), 7.79 (1H, t, J = 8.0 Hz), 7.72 (1H, d, J = 8.4 Hz), 7.62 (1H, t, J = 8.0 Hz), 7.18 (1H, s), 7.08 (1H, s), 6.66 (1H, s), 5.37 (2H, s).

¹³C NMR (100 MHz, CD₃OD): δ 160.7, 159.7, 158.2, 148.2, 139.1, 131.4, 131.3, 129.2, 129.1, 129.0, 128.9, 128.0, 120.6, 110.8, 107.8, 107.0, 71.8.

### Example 2C. Synthesis of 3- (hydroxymethyl) -5- (quinolin-2-ylmethoxy)phenol (CHIN109).

A pure analytical sample (92% yield) was obtained by separating the mixture in HPLC on a Phenomenex pentafluorophenyl C18 reverse phase column and using MeOH/H₂O 60:40 v/v with 0.1% of TFA as eluent mixture (flow at 1 mL/min, t_{R} = 12.24 min).

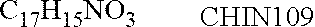

¹H NMR (400 MHz, CDCl₃): δ 8.20 (1H, d, J = 8.5 Hz), 8.10 (1H, d, J = 8.0 Hz), 7.84 (1H, d, J = 8.0 Hz), 7.75 (1H, t, J = 8.0 Hz), 7.67 (1H, d, J = 8.5 Hz), 7.57 (1H, t, J = 8.0 Hz), 6.65 (1H, s), 6.49 (1H, s), 6.46 (1H, s), 5.40 (2H, s), 4.62 (2H, s).

¹³C NMR (100 MHz, CDC1₃): δ 161.1, 159.8, 159.6, 148.3, 145.5, 139.0, 131.3, 129.1, 129.0, 128.9, 127.9, 120.6, 107.8, 105.5, 102.1, 71.6, 65.1.

### Example 3 Synthesis of CHIN111, CHIN112 and CHIN114

To synthesise the compounds CHIN111, CHIN112 and CHIN114, the substituted phenols must first be prepared.

The compound 5 prepared as described in Example 2 step a) by Mitsunobu reaction and by deprotection with TBAF can be transformed into the phenols 6-8, which will then be bound to quinoline by Williamson reaction, in order to obtain the derivatives CHIN110-115.

Diagram 3

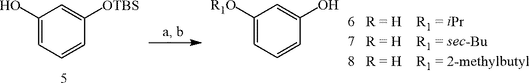

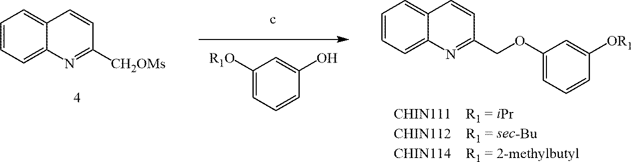

Reagents and conditions: a) PPh₃, DIAD, alcohols of different nature, dry THF, 0° C.; b) tetrabutylammonium fluoride (TBAF) 1.0 M in dry THF, over-night; c) phenols (compounds 6-8), K₂CO₃, anhydrous DMF, 100° C.

### Example 3A. Synthesis of 2-((3-isopropoxyphenoxy)methyl)quinoline (CHIN111).

The purification of CHIN111 (61%) is performed in HPLC on a direct-phase semi-preparative Nucleodur 100-5 column (5 µm; 10 mm i.d. x 250 mm), and using hexane/EtOAc 95:5 v/v as eluent mixture (flow at 3 mL/min, t_{R} = 38 min) .

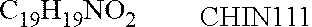

¹H NMR (400 MHz, CDCl₃) : δ 8.19 (1H, d, J = 8.6 Hz), 8.09 (1H, d, J = 7.5 Hz), 7.84 (1H, d, J = 7.5 Hz), 7.74 (1H, t, J = 7.5 Hz), 7.68 (1H, d, J = 8.6 Hz), 7.56 (1H, t, J = 7.5 Hz), 7.17 (1H, t, J = 8.0 Hz), 6.61 (1H, s), 6.60 (1H, ovl), 6.52 (1H, dd, J = 8.0, 2.0 Hz),5.38 (2H, s), 4.52 (1H, heptect, J = 6.0 Hz), 1.32 (6H, d, J = 6.0 Hz).

¹³C NMR (100 MHz, CDCl₃) : δ 159.6, 159.2, 157.9, 147.5, 136.9, 129.9, 129.7, 128.9, 127.7, 127.6, 126.4, 119.1, 108.8, 106.8, 103.0, 71.3, 69.9, 22.0 (2C).

### Example 3B. Synthesis of 2-((3-(sec-butoxy)phenoxy)methyl)quinoline (CHIN112).

The purification is performed in HPLC on a direct-phase semi-preparative Nucleodur 100-5 column (5 µm; 10 mm i.d. x 250 mm), and using hexane/EtOAc 9:1 v/v as eluent mixture (flow at 3 mL/min, t_{R} = 15 min). The compound with quantitative yield is obtained.

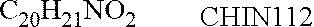

¹H NMR (400 MHz, CDCl₃) : δ 8.19 (1H, d, J = 8.6 Hz), 8.09 (1H, d, J = 7.5 Hz), 7.84 (1H, d, J = 7.5 Hz), 7.74 (1H, t, J = 7.5 Hz), 7.68 (1H, d, J = 8.6 Hz), 7.56 (1H, t, J = 7.5 Hz), 7.17 (1H, t, J = 8.0 Hz), 6.61 (1H, s), 6.60 (1H, ovl), 6.52 (1H, dd, J = 8.0, 2.0 Hz), 5.37 (2H, s), 4.27 (2H, sextet, J = 6.1 Hz), 1.73 (1H, m), 1.60 (1H, m), 1.27 (2H, d, J = 6.1 Hz), 0.96 (3H, t, J = 7.4 Hz).

¹³C NMR (100 MHz, CDCl₃) : δ 159.7, 159.6, 158.0, 147.5, 136.9, 129.9, 129.7, 128.9, 127.7, 127.6, 126.4, 119.2, 108.9, 106.7, 103.1, 75.2, 71.1, 29.2, 19.2, 9.9.

### Example 3C. Synthesis of 2-((3-(2-methylbutoxy)phenoxy)methyl)quinoline (CHIN114).

The purification is performed in HPLC on a direct-phase semi-preparative Nucleodur 100-5 column (5 µm; 10 mm i.d. × 250 mm), and using hexane/AcOEt 9:1 v/v as eluent mixture (flow at 3 mL/min, t_{R} = 14 min). CHIN114 is obtained with 90% yield.

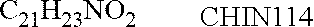

¹H NMR (400 MHz, CDC1₃): δ 8.19 (1H, d, J = 8.6 Hz), 8.09 (1H, d, J = 7.5 Hz), 7.84 (1H, d, J = 7.5 Hz), 7.74 (1H, t, J = 7.5 Hz), 7.68 (1H, d, J = 8.6 Hz), 7.56 (1H, t, J = 7.5 Hz), 7.17 (1H, t, J = 8.0 Hz), 6.61 (1H, s), 6.60 (1H, ovl), 6.52 (1H, dd, J = 8.0, 2.0 Hz), 5.38 (2H, s), 3.80 (1H, dd, J = 9.0, 6.0 Hz), 3.71 (1H, dd, J = 9.0, 6.6 Hz), 1.85 (1H, heptect, J = 6.6 Hz), 1.56 (1H, m), 1.25 (1H, m), 1.00 (3H, d, J = 6.6 Hz), 0.94 (3H, t, J = 7.3 Hz).

¹³C NMR (1 00 MHz, CDC1₃): δ 160.6, 159.6, 158.0, 147.5, 136.9, 129.9, 129.8, 128.9, 127.8, 127.6, 126.5, 119.1, 107.7, 106.8, 101.8, 73.0, 71.2, 34.6, 26.1, 16.5, 11.3.

### Example 4 Synthesis of CHIN116-CHIN121

The esters CHIN116 and CHIN119 are synthesised by Williamson synthesis, using the same experimental process as in example 2 reaction c, starting from 2-(chloromethyl) quinoline (9) and alternatively from methyl 4′-hydroxy-[1,1′-biphenyl]-3-carboxylate (10) or methyl 4′-hydroxy-[1,1′-biphenyl]-4-carboxylate (11).

The esters are then subjected to reduction and hydrolysis, following the experimental processes described earlier in example 1 step a) and step c). Diagram 4.

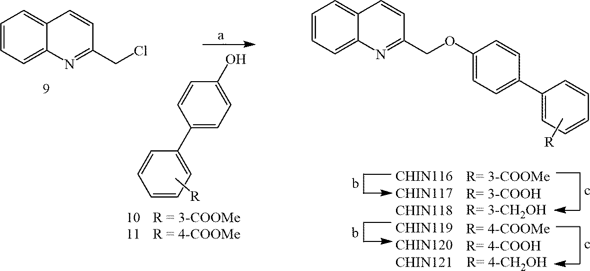

Reagents and conditions. a) compounds 10 or 11, K₂CO₃, dry DMF, 100° C., quantitative yield and 87%, respectively; b) excess NaOH, MeOH: H₂O 1:1 v/v, to be refluxed, quantitative yield for both reactions; c) DIBAL-H, dry THF, 0° C., quantitative and 92% yield, respectively.

### Example 4A. Synthesis of Methyl 4′-(quinolin-2-ylmethoxy)-[1,1′-biphenyl]-3-carboxylate (CHIN116).

The derivative CHIN116 (quantitative yield) is purified on a chromatographic column on silica in hexane: EtOAc 9:1 v/v. An analytical sample is obtained in HPLC on a Nucleodur 100-5 column (5 µm; 10 mm i.d. x 250 mm) using a hexane mixture as eluent: EtOAc 7:3 v/v (flow 3 mL/min, t_{R} = 12.1 min) .

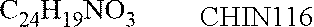

¹H NMR (400 MHz, CDCl₃): δ 8.23 (1H, t, J = 2.0 Hz), 8.21 (1H, d, J = 8.4 Hz), 8.11 (1H, d, J = 8.6 Hz), 7.98 (1H, d, J = 8.0 Hz), 7.84 (1H, d, J = 7.9 Hz), 7.77 (1H, t, J = 8.6 Hz), 7.73 (1H, d, J = 8.4 Hz), 7.71 (1H, d, J = 8.0 Hz), 7.57 (1H, t, ovl), 7.57 (2H, d, J = 8.7 Hz), 7.48 (1H, t, J = 8.0 Hz), 7.13 (2H, d, J = 8.7 Hz), 5.45 (2H, s), 3.94 (3H, s).

¹³C NMR (100 MHz, CDCl₃): δ 167.0, 158.2, 157.7, 147.5, 140.8, 137.1, 133.1, 131.0, 130.6, 129.8, 128.9, 128.8, 128.3 (2C), 127.8 (2C), 127.7, 127.6, 126.5, 119.1, 115.3 (2C), 71.4, 52.1.

### Example 4B. Synthesis of 4′-(quinolin-2-ylmethoxy)-[1,1′-biphenyl]-3-carboxylic Acid (CHIN117).

The compound CHIN117 is obtained in a quantitative yield after purification on a silica chromatographic column (DCM: MeOH 95:5 v/v) .

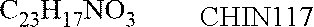

¹H NMR (400 MHz, CDCl₃): δ 8.29 (1H, t, J = 1.6 Hz), 8.23 (1H, d, J = 8.5 Hz), 8.14 (1H, d, J = 8.4 Hz), 8.03 (1H, d, J = 7.8 Hz), 7.85 (1H, d, J = 8.0 Hz), 7.79 (1H, d, J = 7.8 Hz), 7.77 (1H, t, J = 8.4 Hz), 7.72 (1H, d, J = 8.5 Hz), 7.58 (2H, d, J = 8.4 Hz), 7.57 (1H, t, ovl), 7.52 (1H, t, J = 7.8 Hz), 7.14 (2H, d, J = 8.4 Hz), 5.47 (2H, s).

¹³C NMR (100 MHz, DMSO-d6): 168.3, 159.0, 158.5, 147.9, 141.0, 138.2, 133.1, 132.4, 131.0, 130.3, 129.5, 129.0, 128.9, 128.6, 128.3, 127.8, 127.7, 127.6, 120.6, 116.5 (2C), 71.9.

### Example 4C. Synthesis of 4′-(quinolin-2-ylmethoxy)-[1,1′-biphenyl]-3-yl)methanol (CHIN118).

The purification in HPLC on a Nucleodur 100-5 column (5 µm; 10 mm i.d. x 250 mm) with hexane/EtOAc 1:1 v/v as eluent (flow 3 mL/min, t_{R} = 20 min) provided us with CHIN118 in quantitative yield.

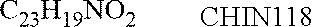

¹H NMR (400 MHz, CDCl₃) : δ 8.22 (1H, d, J = 8.6 Hz), 8.12 (1H, d, J = 7.8 Hz), 7.85 (1H, d, J = 7.8 Hz), 7.76 (1H, t, J = 7.8 Hz), 7.71 (1H, d, J = 8.6 Hz), 7.57 (1H, t, J = 7.7 Hz), 7.55 (1H, s), 7.52 (2H, d, J = 8.6 Hz), 7.47 (1H, d, J = 7.6 Hz), 7.40 (1H, t, J = 7.6 Hz), 7.31 (1H, d, J = 7.6 Hz), 7.09 (2H, d, J = 8.6 Hz), 5.42 (2H, s), 4.76 (2H, s) . ¹³C NMR (100 MHz, CDCl₃) : δ 158.1, 157.8, 147.5, 141.4, 140.9, 137.1, 133.9, 129.8, 128.9, 128.8, 128.3 (2C), 127.7, 127.6, 126.6, 126.0, 125.3 (2C), 119.1, 115.1 (2C), 71.2, 65.4.

### Example 4D. Synthesis of Methyl 4′-(quinolin-2-ylmethoxy)-[1,1′-biphenyl]-4-carboxylate (CHIN119).

The compound CHIN119 (87% yield) is purified by chromatographic column on silica, using a mixture of hexane:EtOAc 9:1 v/v as eluent. A pure analytical sample is obtained by separating in HPLC on a Nucleodur 100-5 C18 column (5 µm; 4.6 mm i.d. × 250 mm) in gradient (t₀= 60% MeOH-t₂₀ₘᵢₙ⁻ 95% MeOH; flow 1 mL/min, t_{R} = 5.5 min) .

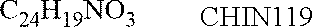

¹H NMR (400 MHz, CDCl₃): δ 8.22 (1H, d, J = 8.6 Hz), 8.11 (1H, d, J = 8.2 Hz), 8.08 (2H, d, J = 8.6 Hz), 7.85 (1H, d, J = 8.2 Hz), 7.76 (1H, t, J = 8.2 Hz), 7.70 (1H, d, J = 8.6 Hz), 7.61 (2H, d J = 8.6 Hz), 7.58 (2H, d, J = 8.9 Hz), 7.57 (1H, t, ovl), 7.13 (2H, d, J = 8.9 Hz), 5.46 (2H, s), 3.94 (3H, s).

¹³C NMR (100 MHz, CDCl₃): δ 167.1, 158.6, 157.6, 147.5, 145.0, 137.1, 132.9, 130.1 (2C), 129.8, 128.9, 128.5 (2C), 128.3, 127.7, 127.6, 126.6 (2C), 126.5, 119.0, 115.3 (2C), 71.5, 52.1.

### Example 4E. Synthesis of 4′-(quinolin-2-ylmethoxy)-[1,1′-biphenyl]-4-carboxylic Acid (CHIN120).

The purification is performed on a flash chromatographic column and silica gel using DCM: MeOH 95:5 v/v as eluent in order to obtain CHIN120 with quantitative yield.

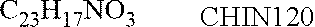

¹H NMR (400 MHz, CD30D+0.1% TFA): δ 9.23 (1H, d, J = 8.5 Hz), 8.42 (1H, d, J = 8.0 Hz), 8.39 (1H, d, J = 7.5 Hz), 8.24 (1H, t, J = 7.5 Hz), 8.23 (1H, d, J = 8.5 Hz), 8.10 (2H, d, J = 8.5 Hz), 8.03 (1H, t, J = 8.0 Hz), 7.76 (2H, d, J = 8.5 Hz), 7.73 (2H, d, J = 8.5 Hz), 7.32 (2H, d, J = 8.5 Hz), 5.80 (2H, s).

¹³C NMR (100 MHz, CDCl₃): δ 168.7, 158.3, 157.3, 145.7, 145.0, 138.8, 133.2, 130.9, 130.4 (2C), 128.5 (2C), 128.4, 128.3, 127.8 (2C), 126.5 (3C), 119.2, 115.3 (2C), 69.8.

### Example 4F. Synthesis of (4′-(quinolin-2-ylmethoxy)-[1,1′-biphenyl]-3-yl)methanol (CHIN121).

The compound CHIN121 is obtained after purification on a silica gel chromatographic column, using hexane as the eluent: EtOAc 8:2 v/v (yield of 92). An analytical sample is further purified in HPLC on a Nucleodur 100-5 column (5 µm; 10 mm i.d. x 250 mm) with hexane/EtOAc 1:1 v/v (flow 3 mL/min, t_{R} = 18 min).

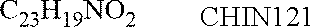

¹H NMR (400 MHz, CDCl₃) : δ 8.22 (1H, d, J = 8.4 Hz), 8.11 (1H, d, J = 8.5 Hz), 7.85 (1H, d, J = 8.0 Hz), 7.76 (1H, t, J = 8.5 Hz), 7.71 (1H, d, J = 8.4 Hz), 7.57 (1H, t, J = 8.0 Hz), 7.55 (2H, d, J = 8.6 Hz), 7.53 (2H, d, J = 8.6 Hz), 7.42 (2H, d, J = 8.6 Hz), 7.10 (2H, d, J = 8.6 Hz), 5.44 (2H, s), 4.74 (2H, s).

¹³C NMR (100 MHz, CDCl₃) : δ 157.9, 157.8, 157.3, 147.5, 140.1, 137.1, 133.9, 129.8, 128.9, 128.5, 128.4, 127.7, 127.6, 127.5 (2C), 126.8 (2C), 126.5, 119.1, 115.2 (2C), 71.3, 65.1.

### Example 5 Synthesis of CHIN125-CHIN127 and CHIN131-CHIN133

For the synthesis of the esters CHIN125 and CHIN131, the first step is to obtain phenols 12 and 13 using the Williamson synthesis, starting with methyl 3,5-dihydroxybenzoate (2) and reacting it alternately with methyl 5-bromopentanoate (14) and methyl 4-bromobutanoate (15). The resulting monoalkylated phenols are subjected to a further Williamson reaction with 2- (chloromethyl) quinoline (9), by the same experimental process as used in example 2 reaction c. The esters are then subjected to reduction with LiBH₄ and hydrolysis, following the experimental process described earlier in example 1 reaction c). Diagram 5.

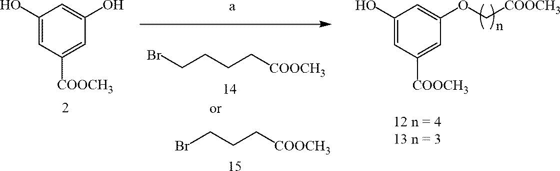

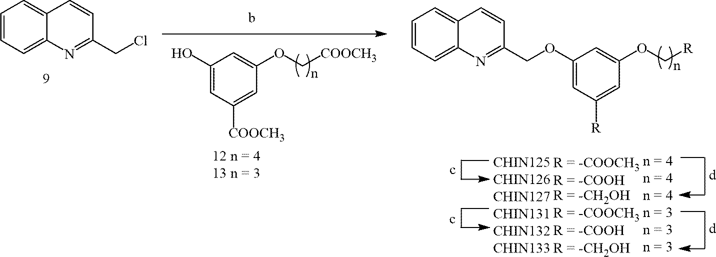

^{a}Reagents and conditions. a) compound 14 or compound 15, K₂CO₃, dry DMF, 100° C., yields 48% and 47%, respectively for the compounds 12 and 13; b) compounds 12 or 13, K₂CO₃, dry DMF, 100° C., yields of 80% and 74%, respectively; c) NaOH in excess, MeOH:H₂O 1:1 v/v, to be refluxed, quantitative yield of 98%, respectively; d) LiBH₄, dry THF, 0° C., yields of 80% and 76%, respectively.

### GENERAL PROCESSES

Reaction a). Williamson’s reaction. Methyl 5-bromopentanoate (0.5 equiv.) or methyl 4-bromobutanoate and K₂CO₃ (1 equiv.) are added to a solution of the compound 2 in dry DMF and the solution is placed at 100° C. for about 12 hours. It is cooled, acidified with HCl 6N and the DMF is removed by rotavapor. The dry residue is extracted with water and ethyl acetate (3 × 50 mL). The pooled organic phases are anhydrified with Na₂SO₄, filtered, and then concentrated on the rotavapor, obtaining a crude reaction product, which will be purified by open column chromatography.

### Reaction D). Reduction Reaction With LiBH₄.

Dry methanol (1 equiv.) and a solution of LiBH₄ 2 M in dry THF (2 equiv.) at 0° C. is added to a solution of the esters CHIN125 or CHIN131 in dry THF. After about 5 h, TLC monitoring of the reaction shows the end of the substrate, the reaction is treated by adding a 1N NaOH solution (2 equiv.) at 0° C. Quenching lasts for 1 h and then the mixture is extracted with water and ethyl acetate (3 × 50 mL). The pooled organic phases are anhydrified with Na₂SO₄, filtered, and then concentrated by rotavapor, obtaining a crude reaction product, which will be purified by means of HPLC.

### Example 5A. Synthesis of Methyl 3-((5-methoxy-5-oxopentyl)oxy)-5-(quinolin-2-ylmethoxy)benzoate (CHIN125).

The compound CHIN125 (80% yield) is purified by chromatographic column on silica, using a hexane mixture as eluent: EtOAc 9:1 v/v. A pure analytical sample is obtained by separating in HPLC on a Nucleodur 100-5 column (5 µm; 10 mm i.d. × 250 mm) with n-hexane: EtOAc 7:3 v/v as eluent (flow rate 3 mL/min, t_{R} = 20.4 min)

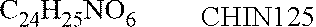

¹H NMR (400 MHz, CDCl₃): δ 8.21 (1H, d, J = 8.5 Hz), 8.10 (1H, d, J = 8.5 Hz), 7.83 (1H, d, J = 8.2 Hz), 7.74 (1H, t, J = 8.5 Hz), 7.66 (1H, d, J = 8.5 Hz), 7.56 (1H, t, J = 8.1 Hz), 7.33 (1H, dd, J = 1.3 Hz, 2.3 Hz), 7.19 (1H, dd, J = 1.3 Hz, 2.3 Hz), 6.78 (1H, t, J = 2.3 Hz), 5.40 (2H, s), 3.98 (2H, t, J = 6.5 Hz), 3.89 (3H, s), 3.67 (3H, s), 2.38 (2H, t, J=7.5 Hz), 1.81 (2H, ovl). ¹³C NMR (700 MHz, CDCl₃) : δ 173.8, 166.7, 160.1, 159.5, 157.3, 147.6, 137.1, 132.1, 129.8, 129.0, 127.7, 127.6, 126.6, 119.1, 108.4, 108.3, 106.7, 71.5, 67.8, 52.3, 51.5, 33.6, 28.5, 21.5.

### Example 5B. Synthesis of 3-(4-carboxybutoxy)-5-(quinolin-2-ylmethoxy)benzoic acid (CHIN126).

The purification is performed by means of HPLC on a Phenomenex Luna C18(2) column (5 µm; 10 mm i.d. × 250 mm) in gradient (t₀= 10% MeCN 0.1% TFA - t_{20 min}= 70% MeCN 0.1% TFA -t_{25 min}= 95% MeCN 0.1% TFA; flow 3 mL/min, t_{R} = 16.3 min) providing the compound CHIN126 (quantitative yield).

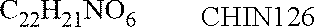

¹H NMR (400 MHz, CD₃OD): δ 9.17 (1H, d, J = 8.5 Hz), 8.38 (1H, d, J = 8.5 Hz), 8.34 (1H, d, J = 8.2 Hz), 8.20 (1H, t, J= 8.5 Hz), 8.18 (1H, d, J = 8.5 Hz), 7.98 (1H, t, J = 8.1 Hz), 7.38 (1H, dd, J = 1.3 Hz, 2.3 Hz), 7.31 (1H, dd, J = 1.3 Hz, 2.3 Hz), 6.98 (1H, t, J = 2.3 Hz), 5.74 (2H, s), 4.06 (2H, t, J = 5.5 Hz), 2.38 (2H, t, J = 8.0 Hz), 1.82 (2H, ovl). ¹³C NMR (700 MHz, CD₃OD): δ 177.3, 169.2, 161.8, 160.4, 157.9, 143.6, 133.5, 132.1, 129.7, 129.5, 129.4, 129.1, 125.9, 121.1, 109.2, 108.9, 107.1, 70.0, 68.9, 34.5, 29.6, 22.7.

### Example 5C. Synthesis of 5-(3-(hydroxymethyl)-5-(quinolin-2-ylmethoxy)phenoxy)pentan-1-ol (CHIN127).

The purification is performed by means of HPLC on a Phenomenex Luna C18(2) column (5 µm; 10 mm i.d. × 250 mm) with MeCN/H₂O 55:45 (flow 3 mL/min, t_{R} = 5.16 min) providing the compound CHIN127 (80%).

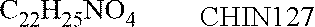

¹H NMR (400 MHz, CDCl₃): δ 8.21 (1H, d, J = 8.5 Hz), 8.11 (1H, d, J = 8.5 Hz), 7.84 (1H, d, J = 8.2 Hz), 7.75 (1H, t, J = 8.5 Hz), 7.68 (1H, d, J = 8.5 Hz), 7.56 (1H, t, J = 8.1 Hz), 6.64 (1H, dd, J = 1.3 Hz, 2.3 Hz), 6.55 (1H, t, J = 2.3 Hz), 6.52 (1H, dd, J = 1.3 Hz, 2.3 Hz), 5.38 (2H, s), 4, 62 (2H, s), 3.94 (2H, t, J = 6.5 Hz), 3.67 (2H, t, J = 6.5), 1.79 (2H, quint. J = 6.2 Hz, 7.5 Hz), 1.63-1.53 (4H, ovl). ¹³C NMR (700 MHz, CDCl₃): δ 160.3, 159.6, 157.8, 147.2, 143.9, 137.3, 130.0, 129.2, 127.7, 127.6, 126.9, 119.3, 105.9, 105.1, 100.8, 71.9, 67.9, 64.8, 62.5, 32.3, 28.9, 22.3.

### Example 5D. Synthesis of Methyl 3-(4-methoxy-4-oxobutoxy)-5-(quinolin-2-ylmethoxy)benzoate (CHIN131).

The derivative CHIN131 (74%) is purified on a silica chromatographic column in hexane:EtOAc 9:1 v/v. An analytical sample is obtained in HPLC on a Nucleodur 100-5 column (5 µm; 10 mm i.d. × 250 mm) using a mixture of n-hexane:EtOAc 7:3 v/v as eluent (flow 3 mL/min, t_{R} = 22.1 min).

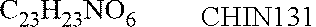

¹H NMR (400 MHz, CDCl₃): δ 8.24 (1H, d, J = 8.5 Hz), 8.13 (1H, d, J = 8.5 Hz), 7.85 (1H, d, J = 8.2 Hz), 7.76 (1H, t, J = 8.5 Hz), 7.68 (1H, d, J = 8.5 Hz), 7.58 (1H, t, J = 8.1 Hz), 7.34 (1H, dd, J = 8.1 Hz, 2.3 Hz), 7.19 (1H, dd, J = 2.3 Hz), 6.78 (1H, dd, J = 8.1 Hz, 2.3 Hz), 5.38 (2H, s), 4.02 (2H, t, J = 6.3 Hz), 3.71 (2H, t, J = 7.0 Hz), 2.51 (2H, t, J = 7.3 Hz), 2.10 (2H, quint., J = 6.3 Hz, 7.3 Hz). ¹³C NMR (700 MHz, CDCl₃): δ 173.6, 166.7, 160.3, 159.6, 157.3, 147.6, 137.2, 132.2, 129.9, 129.1, 127.8, 127.7, 126.7, 119.2, 108.5, 108.3, 106.8, 71.5, 67.1, 52.3, 51.8, 30.5, 24.5.

### Example 5E. Synthesis of 3-(3-carboxypropoxy)-5-(quinolin-2-ylmethoxy)benzoic Acid (CHIN132).

The derivative CHIN132 (98%) is purified by means of HPLC on a Phenomenex Luna C18(2) column (5 µm; 10 mm i.d. × 250 mm) in gradient (t₀= 10% MeCN 0.1% TFA - t_{20 min}= 70% MeCN 0.1% TFA - t_{25 min}= 95% MeCN 0.1% TFA; flow 3 mL/min, t_{R} = 15 min) .

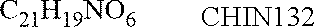

¹H NMR (400 MHz, CD₃OD): δ 9.04 (1H, d, J = 8.5 Hz), 8.33 (1H, d, J = 8.5 Hz), 8.29 (1H, d, J = 8.2 Hz), 8.14 (1H, t, J = 8.5 Hz), 8.11 (1H, d, J = 8.5 Hz), 7.93 (1H, t, J = 8.1 Hz), 7.38 (1H, dd, J = 8.1 Hz, 2.3 Hz), 7.28 (1H, dd, J = 2.3 Hz), 6.99 (1H, dd, J = 8.1 Hz, 2.3 Hz), 5.69 (2H, s), 4.09 (2H, t, J = 6.3 Hz), 2.50 (2H, t, J = 7.3 Hz), 2.08 (2H, quint., J = 6.3 Hz, 7.3 Hz). ¹³C NMR (700 MHz, CD₃OD) : δ 176.9, 169.0, 161.3, 160.2, 159.7, 148.1, 133.7, 132.0, 130.1, 129.5, 129.4, 129.1, 124.7, 121.3, 109.3, 107.6, 107.5, 69.6, 68.8, 33.0, 25.7.

### Example 5F. Synthesis of 4-(3-(hydroxymethyl)-5-(quinolin-2-ylmethoxy)phenoxy)butan-1-ol (CHIN133).

The derivative CHIN133 (76%) is purified by means of HPLC on a Nucleodur 100-5 column (5 µm; 10 mm i.d. × 250 mm) using a mixture of n-hexane:EtOAc 4:6 v/v as eluent (flow 3 mL/min, t_{R} = 26.32 min)

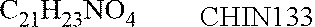

¹H NMR (400 MHz, CD₃OD) : δ 8.23 (1H, d, J = 8.5 Hz), 8.12 (1H, d, J = 8.5 Hz), 7.85 (1H, d, J = 8.2 Hz), 7.76 (1H, t, J = 8.5 Hz), 7.69 (1H, d, J = 8.5 Hz), 7.57 (1H, t, J = 8.1 Hz), 6.65 (1H, dd, J = 8.1 Hz, 2.3 Hz), 6.55 (1H, dd, J = 8.1 Hz, 2.3 Hz), 6.52 (1H, dd, J = 2.3 Hz), 5.39 (2H, s), 4.64 (2H, s), 4.07 (2H, t, J= 6.3 Hz), 3.86 (2H, t, J= 6.3 Hz), 2.49 (2H, m), 2.02 (2H, m) . ¹³C NMR (700 MHz, CD₃OD) : δ 176.9, 169.0, 161.3, 160.2, 159.7, 148.1, 133.7, 132.0, 130.1, 129.5, 129.4, 129.1, 124.7, 121.3, 109.3, 107.6, 107.5, 69.6, 68.8, 33.0, 25.7.

### Example 6 Synthesis of CHIN134-CHIN142

The first reaction step is the Mitsunobu reaction, the process of which is described in example 1 step b), starting with methyl 3,5-dihydroxybenzoate (2) with propan-2-ol (16), propan-1-ol (17) and butan-2-ol(18). The resulting monoalkylated derivatives are subjected to a Williamson reaction with 2-(chloromethyl)quinoline (9), using the same experimental process as used in example 2 reaction c. The esters are then subjected to reduction and hydrolysis, following the experimental processes described earlier in example 5 reaction d) and example 1 reaction c), respectively.

Diagram 6.

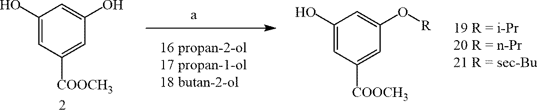

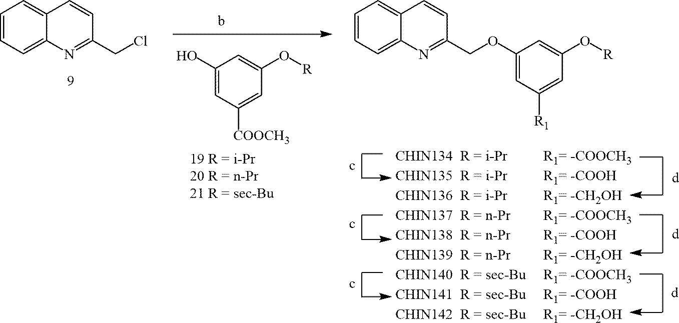

^{a}Reagents and conditions. a) alcohol 16 or 17 or 18, DIAD, PPh₃, dry THF, 0° C., quantitative yields, 50%, 45% and 42%, respectively for the compounds 19, 20 and 21; b) compounds 19-21, K₂CO₃, dry DMF, 100° C., quantitative yields, 57% and 70%, respectively; c) NaOH in excess, MeOH:H₂O 1:1 v/v, to be refluxed, yields of 84%, 86% and 91%, respectively; d) LiBH₄, dry THF, 0° C., yields of 88%, 94% and 89%, respectively.

### Example 6A. Synthesis of Methyl 3-isopropoxy-5-(quinolin-2-ylmethoxy)benzoate (CHIN134).

The derivative CHIN134 (50%) is purified on a chromatographic column on silica in hexane: EtOAc 9:1 v/v. An analytical sample is obtained in HPLC on a Nucleodur 100-5 column (5 µm; 10 mm i.d. × 250 mm) using a mixture of n-hexane:EtOAc 7:3 v/v as eluent (flow 3 mL/min, t_{R} = 9.9 min).

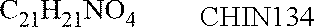

¹H NMR (400 MHz, CDCl₃): δ 8.21 (1H, d, J = 8.5 Hz), 8.10 (1H, d, J = 8.5 Hz), 7.84 (1H, d, J = 8.2 Hz), 7.75 (1H, t, J = 8.5 Hz), 7.67 (1H, d, J = 8.5 Hz), 7.56 (1H, t, J = 8.1 Hz), 7.31 (1H, dd, J = 2.4 Hz), 7.20 (1H, dd, J = 2.3 Hz), 6.78 (1H, dd, J = 2.4 Hz, 2.3 Hz), 5.40 (2H, s), 4.57 (1H, quint., J= 6.1 Hz), 3.89 (3H, s), 1.32 (6H, d, J = 6.1 Hz). ¹³C NMR (700 MHz, CDCl₃): δ 166.8, 159.5, 159.1, 157.3, 147.6, 137.1, 132.2, 129.9, 129.0, 127.8, 127.7, 126.7, 119.2, 109.7, 108.2, 108.0, 71.4, 70.4, 52.1, 21.9(2C).

### Example 6B. Synthesis of 3-isopropoxy-5-(quinolin-2-ylmethoxy)benzoic Acid (CHIN135).

The derivative CHIN135 (84%) is purified on a silica chromatographic column in DCM:MeOH 9:1 v/v.

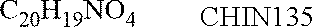

¹H NMR (400 MHz, CD₃OD): δ 9.21 (1H, d, J = 8.5 Hz), 8.39 (1H, d, J = 8.5 Hz), 8.37 (1H, d, J = 8.2 Hz), 8.22 (1H, t, J = 8.5 Hz), 8.20 (1H, d, J = 8.5 Hz), 8.01 (1H, t, J = 8.1 Hz), 7.38 (1H, dd, J = 2.4 Hz), 7.29 (1H, dd, J = 2.3 Hz), 6.97 (1H, dd, J = 2.4 Hz, 2.3 Hz), 5.75 (2H, s), 4.68 (1H, quint., J = 6.1 Hz), 1.35 (6H, d, J = 6.1 Hz). ¹³C NMR (700 MHz, CD₃OD): δ 169.0, 160.7, 159.8, 156.8, 148.6, 136.6, 134.4, 131.2, 130.5, 129.9, 129.2, 126.2, 121.7, 111.8, 109.1, 108.8, 71.6, 67.7, 22.1(2C).

### Example 6C. Synthesis of (3-isopropoxy-5-(quinolin-2-ylmethoxy)phenyl)methanol (CHIN136).

The derivative CHIN136 (88%) is purified by means of HPLC on a Nucleodur 100-5 column (5 µm; 10 mm i.d. × 250 mm) using a mixture of n-hexane:EtOAc 7:3 v/v as eluent (flow 3 mL/min, t_{R} = 38.79 min)

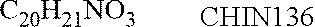

¹H NMR (400 MHz, CDCl₃): δ 8.21 (1H, d, J = 8.5 Hz), 8.12 (1H, d, J = 8.5 Hz), 7.84 (1H, d, J = 8.2 Hz), 7.75 (1H, t, J = 8.5 Hz), 7.68 (1H, d, J = 8.5 Hz), 7.56 (1H, t, J = 8.1 Hz), 6.63 (1H, dd, J = 2.4 Hz), 6.54 (1H, dd, J = 2.3 Hz), 6.51 (1H, dd, J = 2.4 Hz, 2.3 Hz), 5.38 (2H, s), 4.61 (2H, s), 4.52 (1H, quint., J= 6.1 Hz), 1.30 (6H, d, J= 6.1 Hz).

¹³C NMR (700 MHz, CDCl₃): δ 159.8, 159.4, 157.9, 147.5, 143.9, 137.2, 129.9, 129.3, 127.8, 127.7, 127.8, 127.7, 119.3, 107.3, 105.3, 102.2, 70.1, 65.2, 50.6, 22.1(2C).

### Example 6D. Synthesis of Methyl 3-propoxy-5-(quinolin-2-ylmethoxy)benzoate (CHIN137).

The derivative CHIN137 (50%) is purified on a silica chromatographic column in hexane:EtOAc 9:1 v/v. An analytical sample is obtained in HPLC on a Nucleodur 100-5 column (5 µm; 10 mm i.d. × 250 mm) using a mixture of n-hexane:EtOAc 7:3 v/v as eluent (flow 3 mL/min, t_{R} = 11.44 min) .

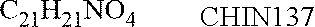

¹H NMR (400 MHz, CDCl₃): δ 8.21 (1H, d, J = 8.5 Hz), 8.10 (1H, d, J = 8.5 Hz), 7.84 (1H, d, J = 8.2 Hz), 7.75 (1H, t, J = 8.5 Hz), 7.67 (1H, d, J = 8.5 Hz), 7.56 (1H, t, J = 8.1 Hz), 7.33 (1H, dd, J = 2.4 Hz), 7.20 (1H, dd, J = 2.3 Hz), 6.80 (1H, dd, J = 2.4 Hz, 2.3 Hz), 5.40 (2H, s), 3.93 (2H, t, J = 6.4 Hz), 3.89 (3H, s), 1.79 (2H, sext., J = 7.4 Hz), 1.02 (3H, t, J = 7.4 Hz). ¹³C NMR (700 MHz, CDCl₃) : δ 166.9, 160.3, 159.5, 157.5, 147.5, 137.2, 132.2, 129.9, 129.0, 127.7, 127.6, 126.7, 119.2, 108.5, 108.2, 106.8, 71.5, 69.9, 52.3, 22.6, 10.5.

### Example 6E. Synthesis of 3-propoxy-5-(quinolin-2-ylmethoxy)benzoic Acid (CHIN138).

The derivative CHIN135 (86%) is purified on a silica chromatographic column in DCM:MeOH 9:1 v/v.

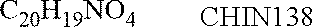

¹H NMR (400 MHz, CD₃OD) : δ 8.40 (1H, d, J = 8.5 Hz), 8.06 (1H, d, J = 8.5 Hz), 7.96 (1H, d, J = 8.2 Hz), 7.80 (1H, t, J = 8.5 Hz), 7.73 (1H, d, J = 8.5 Hz), 7.62 (1H, t, J = 8.1 Hz), 7.28 (1H, dd, J = 2.4 Hz), 7.18 (1H, dd, J = 2.3 Hz), 6.85 (1H, dd, J = 2.4 Hz, 2.3 Hz), 5.39 (2H, s), 3.95 (2H, t, J = 6.4 Hz), 1.78 (2H, sext., J = 7.4 Hz), 1.03 (3H, t, J = 7.4 Hz). ¹³C NMR (700 MHz, CD₃OD) : δ 169.5, 161.7, 160.8, 158.9, 148.3, 139.0, 134.2, 131.2, 129.2, 129.1, 129.0, 128.0, 120.7, 109.7, 109.2, 107.5, 72.0, 70.8, 23.5, 10.7.

### Example 6F. Synthesis of (3-propoxy-5-(quinolin-2-ylmethoxy)phenyl)methanol (CHIN139).

The derivative CHIN139 (94%) is purified by means of HPLC on a Nucleodur 100-5 column (5 µm; 10 mm i.d. × 250 mm) using a mixture of n-hexane:EtOAc 4:6 v/v as eluent (flow 3 mL/min, t_{R} = 24.5 min)

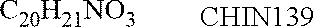

¹H NMR (400 MHz, CD₃OD): δ 8.38 (1H, d, J = 8.5 Hz), 8.05 (1H, d, J = 8.5 Hz), 7.95 (1H, d, J = 8.2 Hz), 7.80 (1H, t, J = 8.5 Hz), 7.73 (1H, d, J = 8.5 Hz), 7.62 (1H, t, J = 8.1 Hz), 6.65 (1H, dd, J = 2.4 Hz), 6.55 (1H, dd, J = 2.3 Hz), 6.51 (1H, dd, J = 2.4 Hz, 2.3 Hz), 5.34 (2H, s), 4.52 (2H, s), 3.90 (2H, t, J = 6.4 Hz), 1.75 (2H, sext., J = 7.4 Hz), 1.01 (3H, t, J = 7.4 Hz). ¹³C NMR (700 MHz, CD₃OD): δ 160.5, 159.6, 158.0, 146.9, 144.1, 137.5, 129.9, 129.0, 127.7, 127.5, 126.5, 119.3, 105.5, 104.9, 100.2, 70.3, 69.2, 63.7, 22.2, 09.4.

### Example 6G. Synthesis of Methyl 3-(sec-butoxy)-5-(quinolin-2-ylmethoxy)benzoate (CHIN140).

The derivative CHIN140 (70%) is purified on a silica chromatographic column in hexane:EtOAc 9:1 v/v. An analytical sample is obtained in HPLC on a Nucleodur 100-5 column (5 µm; 10 mm i.d. × 250 mm) using a mixture of n-hexane:EtOAc 7:3 v/v as eluent (flow 3 mL/min, t_{R} = 11.5 min) .

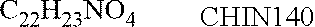

¹H NMR (400 MHz, CDCl₃): δ 8.21 (1H, d, J = 8.5 Hz), 8.10 (1H, d, J = 8.5 Hz), 7.84 (1H, d, J = 8.2 Hz), 7.75 (1H, t, J = 8.5 Hz), 7.67 (1H, d, J = 8.5 Hz), 7.56 (1H, t, J = 8.1 Hz), 7.31 (1H, dd, J = 2.4 Hz), 7.20 (1H, dd, J = 2.3 Hz), 6.78 (1H, dd, J = 2.4 Hz, 2.3 Hz), 5.40 (2H, s), 4.32 (1H, sext. J = 6.0 Hz), 3.88 (3H, s), 1.72-1.61 (2H, ovl), 1.26 (3H, d, J = 6.1 Hz), 0.95 (3H, t, J = 7.4 Hz). ¹³C NMR (700 MHz, CDCl₃): δ 166.9, 159.5, 159.4, 157.3, 147.5, 137.4, 132.5, 130.0, 128.9, 127.8, 127.7, 126.7, 119.2, 109.9, 108.1, 108.0, 75.6, 71.5, 52.3, 19.2, 09.7.

### Example 6H. Synthesis of 3-(sec-butoxy)-5-(quinolin-2-ylmethoxy)benzoic Acid (CHIN141).

The derivative CHIN141 (91%) is purified on a silica chromatographic column in DCM:MeOH 9:1 v/v.

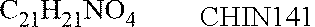

¹H NMR (400 MHz, CD₃OD): δ 8.39 (1H, d, J = 8.5 Hz), 8.06 (1H, d, J = 8.5 Hz), 7.96 (1H, d, J = 8.2 Hz), 7.80 (1H, t, J = 8.5 Hz), 7.72 (1H, d, J = 8.5 Hz), 7.62 (1H, t, J = 8.1 Hz), 7.27 (1H, dd, J = 2.4 Hz), 7.16 (1H, dd, J = 2.3 Hz), 6.83 (1H, dd, J = 2.4 Hz, 2.3 Hz), 5.39 (2H, s), 4.36 (1H, sext. J= 6.0 Hz), 1.69-1.60 (2H, ovl), 1.24 (3H, d, J= 6.1 Hz), 0.96 (3H, t, J = 7.4 Hz). ¹³C NMR (700 MHz, CD₃OD) : δ 169.4, 160.8, 158.8, 148.3, 139.0, 134.0, 131.3, 131.0, 129.2, 129.1, 129.0, 128.0, 120.7, 110.9, 109.2, 108.6, 76.5, 72.0, 30.0, 19.4, 09.9.

### Example 6I. Synthesis of (3-(sec-butoxy)-5-(quinolin-2-ylmethoxy)phenyl)methanol (CHIN142).

The derivative CHIN142 (89%) is purified by means of HPLC on a Nucleodur 100-5 column (5 µm; 10 mm i.d. × 250 mm) using a mixture of n-hexane:EtOAc 4:6 v/v as eluent (flow 3 mL/min, t_{R} = 24.74 min)

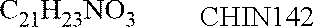

¹H NMR (400 MHz, CDCl₃): δ 8.21 (1H, d, J = 8.5 Hz), 8.12 (1H, d, J = 8.5 Hz), 7.83 (1H, d, J = 8.2 Hz), 7.75 (1H, t, J= 8.5 Hz), 7.68 (1H, d, J = 8.5 Hz), 7.56 (1H, t, J = 8.1 Hz), 6.63 (1H, dd, J = 2.4 Hz), 6.54 (1H, dd, J = 2.3 Hz), 6.51 (1H, dd, J = 2.4 Hz, 2.3 Hz), 5.38 (2H, s), 4.61 (2H, s), 4.27 (1H, sext. J = 6.0 Hz), 1.71-1.59 (2H, ovl), 1.25 (3H, d, J = 6.1 Hz), 0.94 (3H, t, J = 7.4 Hz). ¹³C NMR (700 MHz, CDCl₃): δ 159.8, 159.7, 157.9, 147.4, 143.8, 137.1, 129.9, 128.8, 127.7, 127.6, 126.6, 119.2, 107.3, 105.3, 102.1, 75.1, 71.2, 65.1, 29.1, 19.3, 09.8.

### Example 7 - Biological Data

The activity data of the compounds of the invention on FXR, TGR5/GPBAR1 and CysLT1R receptors are described in Table 1. In this table, the activities of the compounds are compared with specific reference compounds, namely CDCA for FXR, TLCA for TGR5/GPBAR1, MK571 for CysLT1R. Each compound is tested at a concentration of 10 microM and the activity of the reference compounds is considered to be 100%.

TABLE 1

| Compound s of formula (Ia) | FXR (% of activity compared to 10 µM CDCA) | ECso (µM) | GPBAR1 (% of activity compared to 10 µM TLCA) | ECso (µM) | CysLTR1 (% of inhibition compared to the compoundMK571) | ICso (µM) |
|---|---|---|---|---|---|---|
| CHIN104 | 32.46 | 9 | 23 | | 85 | 3.9 |
| CHIN105 | 28 | 30 | 74.8 | 3 | 71 | 2.8 |
| CHIN106 | 21.88 | - | 73.2 | 7.4 | 97 | 1.2 |
| CHIN107 | 12.78 | - | 17.58 | | 59 | >10 |
| CHIN108 | 8.22 | - | 29.8 | 20 | na | - |
| CHIN109 | 5.92 | - | 72.92 | 23 | 26 | - |
| CHIN111 | na | - | na | - | 79 | - |
| CHIN112 | na | - | 100.5 | - | 66 | 9.63 |
| CHIN114 | na | - | 106.43 | 0.17 | 15 | - |
| CHIN116 | 11.14 | - | 56.91 | - | 21 | - |
| CHIN117 | 8.9 | - | 82 | 4.6 | 109 | 5.67 |
| CHIN118 | na | - | na | - | 59 | - |
| CHIN119 | 6.83 | - | 13.5 | - | 13 | - |
| CHIN120 | 12.76 | - | 36.74 | - | 43 | - |
| CHIN121 | 8.22 | - | 64.9 | - | na | - |

For FXR-mediated transactivation, the HepG2 cells were transfected with 200 ng of the p(hsp27)-TK-LUC reporter vector containing the FXR response element (IR1) cloned from the heat shock protein 27 (hsp27) promoter, 100 ng of pSG5-FXR, 100 ng of pSG5-RXR and 100 of pGL4.70 (Promega, Madison WI), a vector encoding for the human Renilla gene.

For GPBAR1-mediated transactivation, the HEK-293T cells were transfected with 200 ng of pGL4.29 (Promega, Madison WI), a reporter vector containing a cAMP response element (CRE) that drives the transcription of the luc2P luciferase reporter gene, with 100 ng of human pCMVSPORT6-GPBAR1, and with 100 ng of pGL4.70.

24 hours after transfection, the cells were stimulated for 18 hours with specific receptor agonists CDCA (10 µM) or TLCA (10 µM) or with the derivatives CHIN104-112 and CHIN114-121 (10 µM and 50 µM). In another experimental set, 24 hours after transfection, the cells were stimulated with 50 µM of the derivatives CHIN in combination with 10 µM of CDCA or TLCA.

For dose-response curves, the cells were stimulated with increasing concentrations of the compounds of interest (0.1-75 µM). Eighteen hours after stimulation, the cell lysates were used to assess Luciferase and Renilla activity by means of the Dual-Luciferase Reporter assay (E1980, Promega Madison WI). The luminescence was measured using the Glomax 20/20 luminometer (Promega, Madison WI) and the Luciferase activity was normalised with the Renilla activity.

The antagonistic activity assay was performed by Eurofins Cerep-Panlabs (France). The cells were suspended in DMEM buffer (Invitrogen), then plated at a density of 3·10⁴ cells/plate. The fluorescent probe (Fluo4 Direct, Invitrogen) mixed with probenicid in HBSS buffer (Invitrogen) supplemented with 20 mM Hepes (Invitrogen) (pH 7.4) is then added to each well and left with the cells for 60 min at 37° C. and then for 15 min at 22° C. The plates are then placed in a microplate reader (CellLux, PerkinElmer), which is used to add the compounds to be tested or the HBSS buffer and then after 5 min with a 0.1 nM LTD4 or HBSS buffer solution (used as control). The change in intensity and fluorescence that varies in proportion to the concentration of free Ca²⁺ ion in the cytosol is measured. The result is expressed as a percentage of inhibition compared to the control response to 0.1 nM LTD4. The standard antagonist reference is MK 571.

CHIN117 and the effect thereof in reducing acetaminophen-induced liver damage (APAP) are of particular interest. In the experimental set, acute hepatitis was induced in wild-type C57/B16 mice by administration of acetaminophen (APAP) at a concentration of 500 mg/kg via oral gavage. 45 minutes after induction of the disease, CHIN117 was administered at a concentration of 30 mg/kg orally. The mice were sacrificed 24 hours after induction of the disease and blood was taken and analysed for blood count and AST and ALT transaminase values.

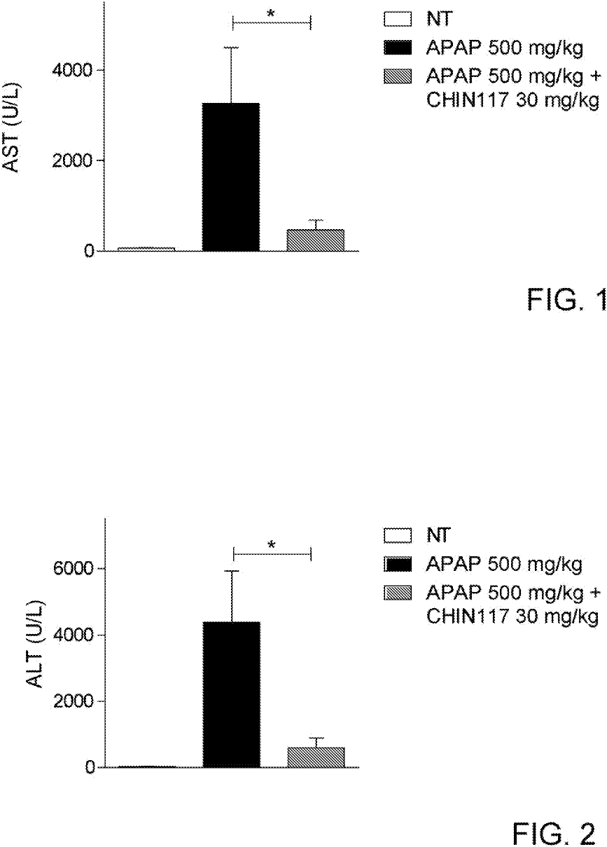

The results show (FIGS. 1-3) that the administration of APAP induces a sharp increase in AST and ALT values (between 3000 and 4000) and, in addition, the hepatic damage produced attracts immune cells involved in the pathogenesis of the disease to the liver, with a consequent decrease in circulating white blood cell (WBC) values. The administration of the compound CHIN117 was able to alleviate the APAP-induced liver damage by reducing AST and ALT values by about 10-fold if compared to those recorded in the group of mice treated with APAP alone. CHIN117 was also able to maintain WBC values comparable to those found in untreated mice (NT).

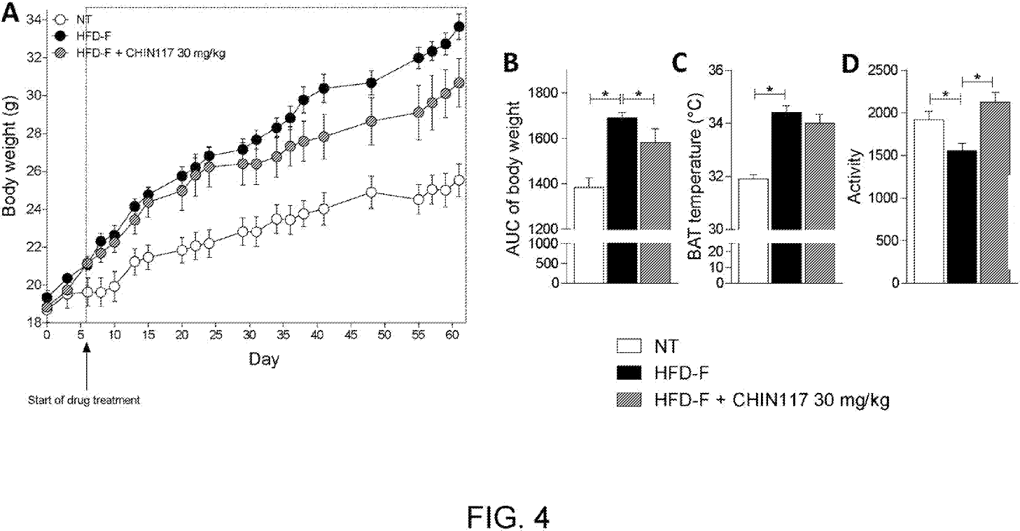

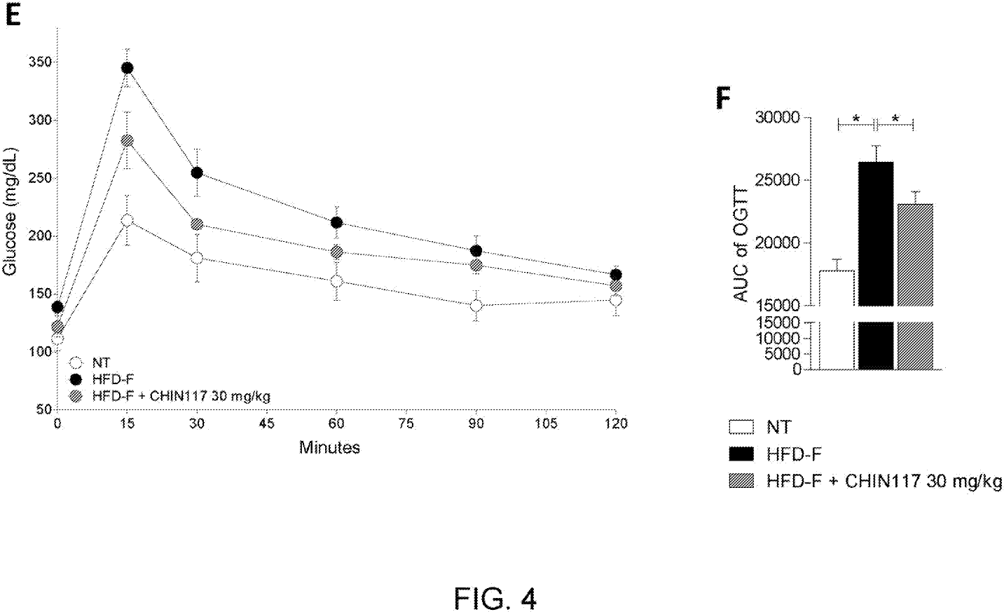

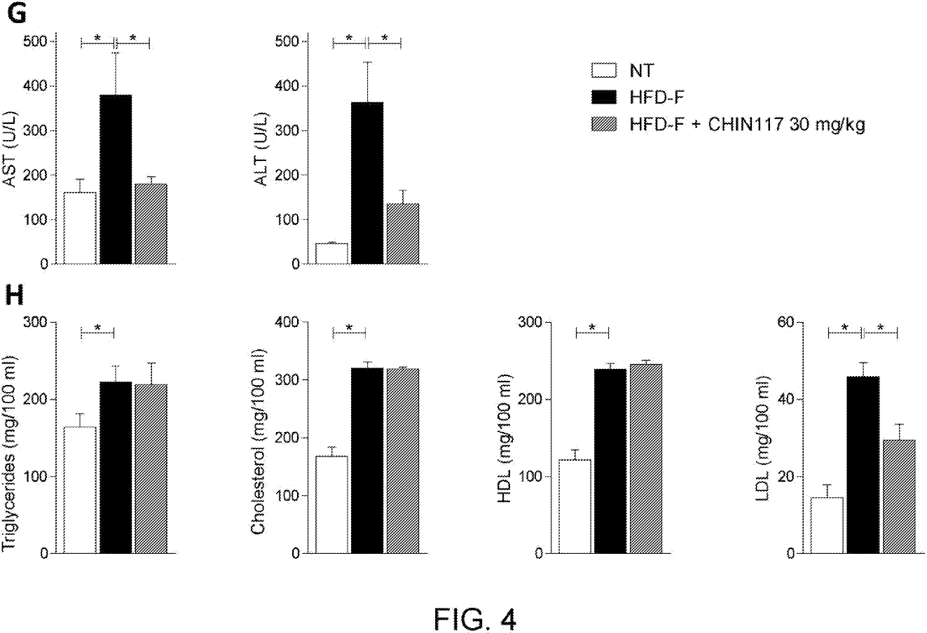

CHIN117 and the efficacy thereof in a murine model of chronic hepatitis induced by a high-fat diet are of particular interest. This murine model simulates NAFLD, which represents a rapidly growing epidemic in industrialised countries and has a very high cost for the health care system. In this model, the mice (male C57BL/6 mice) were fed a diet rich in lipids and cholesterol (2% cholesterol) and added fructose (3%) in water (HFD-F) for 60 days. CHIN117 was administered daily at a dose of 30 mg/kg starting on day 7. The weight trend shows that CHIN117 reduces weight gain by about 3 grams (FIGS. 4A, B). After eight weeks, the mice develop insulin resistance, as shown by the OGTT results (FIGS. 4E, F). The treatment of the mice with CHIN117 reverses the effect of the diet and reduces the AUC of OGTT. In addition, CHIN117 statistically reduces AST, ALT and LDL levels counteracting the hepatotoxic effect of the HFD-F diet (FIGS. 4G-H).

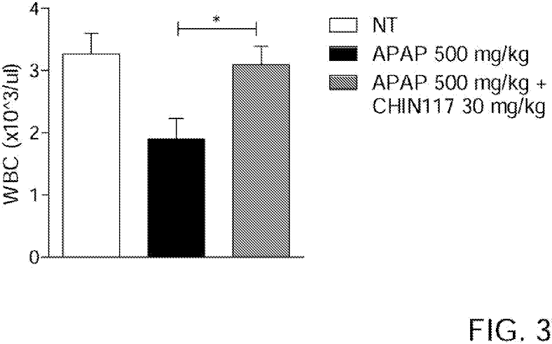

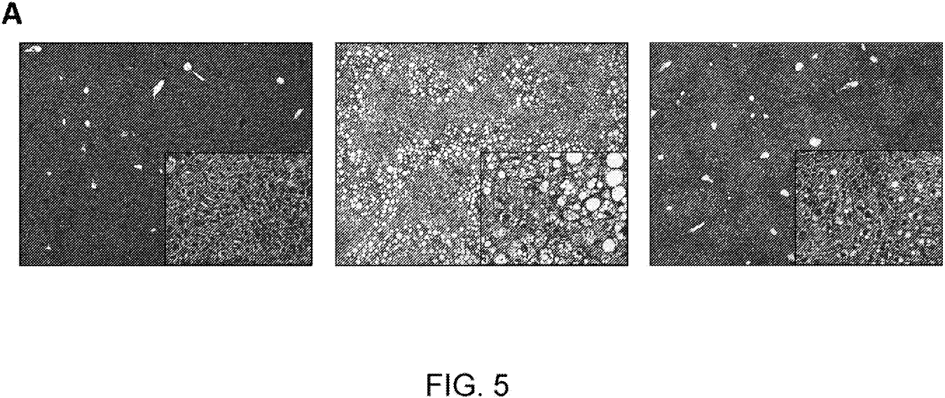

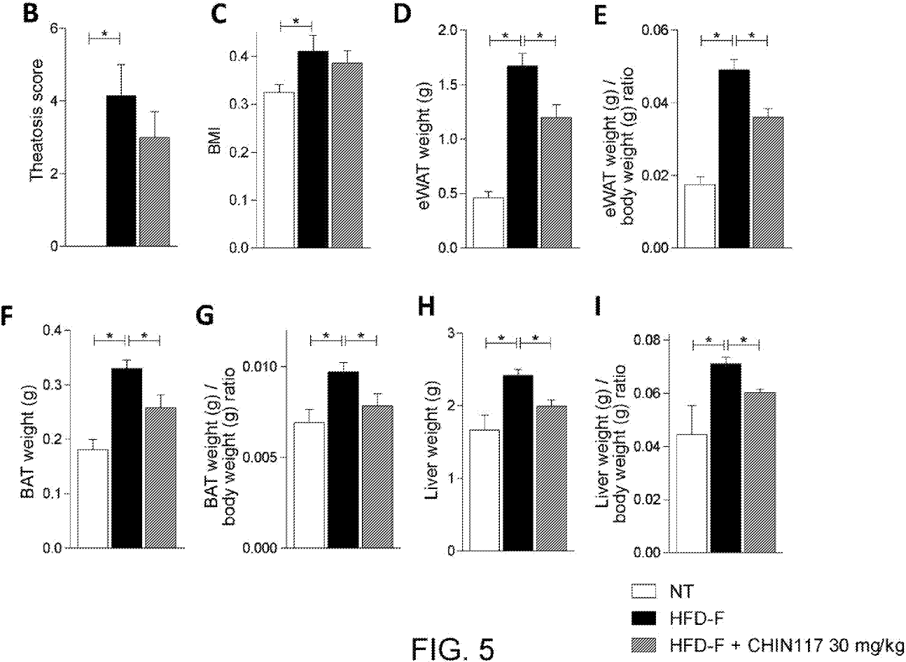

The mice fed an HFD-F diet for 8 weeks develop characteristics similar to human NASH as revealed by the H&E staining in liver sections, with micro-vesicular steatosis, swelling of hepatocytes, lobular inflammation and influx of macrophages (FIG. 5A) leading to a significant increase in the hepatic steatosis (NAS) score (FIG. 3B). In addition, HFD-F indicates an increase in body mass index (BMI) and weight of the white adipose tissue of the epididymis (eWAT), of the brown adipose tissue (BAT) and of the liver (FIGS. 5C-I). CHIN117 almost completely reversed the disease by reducing hepatic steatosis, BMI and eWAT, BAT and liver weight (FIG. 5).

CHIN117 is of particular interest for its excellent pharmacokinetic properties with an aqueous solubility at pH 7.4 equal to 66 µM and a LogD =2.0 and for its promising metabolic stability upon exposure in vitro to microsomal enzymes at t_{½} = 578 min (CLᵢₙₜ = 4) and to the S9 fraction, which also contains enzymes responsible for phase II metabolism, at t_{½} = 385 min (CLᵢₙₜ = 6).

1. A compound of formula (I): 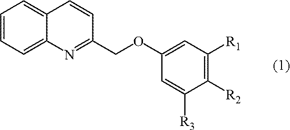
or pharmaceutically acceptable salts or solvates thereof wherein:
R₁ is selected from the group consisting of H, linear or branched C₁₋₆ alkyl optionally substituted with one substituent R₇; linear or branched O-C₃₋₆ alkyl optionally substituted with one substituent R₈;
R₂ is selected from the group consisting of H, C₁₋₆ hydroxyalkyl, linear or branched C₁₋₆ alkyl optionally substituted with one substituent R₇, phenyl optionally substituted with at least one substituent independently selected from the group consisting of COOH, COO-C₁₋₆ alkyl, C₁₋₆ hydroxyalkyl and linear or branched C₁₋₆ alkyl optionally substituted with one substituent R₉;
R₃ is selected from the group consisting of H, COOH, COO-C₁₋₆ alkyl, CH₂OH; provided that:
when R₂ is H, R₁ or R₃ are not H; or
when R₁ is linear O-C₃₋₆ alkyl optionally substituted with one substituent R₈, at least one between R₂ and R₃ is not H;
R₄ is selected from the group consisting of H, COOH, COO-C₁₋₆ alkyl, C₁₋₆ hydroxyalkyl;
R₅ is selected from the group consisting of H, COOH, COO-C₁₋₆ alkyl, C₁₋₆ hydroxyalkyl;
R₆ is selected from the group consisting of H, COOH, COO-C₁₋₆ alkyl, C₁₋₆ hydroxyalkyl, OH and linear or branched C₁₋₆ alkoxyl optionally substituted with one substituent R₈;
provided that when one between R₄, R₅ and R₆ is COOH or CH₂OH, at least one of the remaining ones between R₄, R₅ and R₆ is not H,
R₇ is selected from the group consisting of: 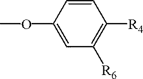 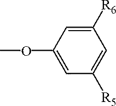
R
₈ is selected from the group consisting of OH, COOH, COO-C₁₋₆ alkyl;
R₉ is selected from the group consisting of: 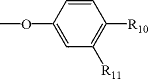 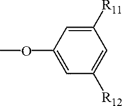
where R
₁₀ is selected from the group consisting of H, COOH, COO-C₁₋₆ alkyl, C₁₋₆ hydroxyalkyl;
R₁₁ is selected from the group consisting of H, OH, COOH, COO-C₁₋₆ alkyl, C₁₋₆ hydroxyalkyl;
and R₁₂ is selected from the group consisting of H, COOH, COO-C₁₋₆ alkyl, C₁₋₆ hydroxyalkyl; excluded the compounds: 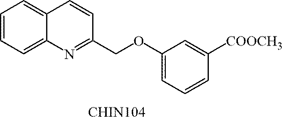 [SMILES: COC(=O)c1cccc(OCc2ccc3ccccc3n2)c1 ; "CHIN104"] 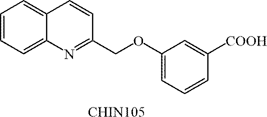 [SMILES: O=C(O)c1cccc(OCc2ccc3ccccc3n2)c1 ; "CHIN105"] 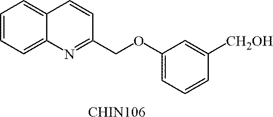 [SMILES: OCc1cccc(OCc2ccc3ccccc3n2)c1 ; "CHIN106"]
.

2. The compound of formula (I) according to claim 1, wherein R₁ is selected from the group consisting of H, O-i-propyl, O-n-propyl, O-sec-butyl, O-n-pentyl, O-2-methylbutyl, -CH₂-R₇, —O—(CH₂)₃₋₄-R₈.

3. The compound of formula (I) according to claim 1, wherein R₂ is a phenyl optionally substituted with at least one substituent independently selected from the group consisting of H, COOH, COO-C₁₋₆ alkyl, C₁₋₆ hydroxyalkyl and linear or branched C₁₋₆ alkyl optionally substituted with one substituent R₉.

4. The compound of formula (I) according to claim 1, wherein R₂ is selected from the group consisting of H, CH₂OH, CH₂-R₇, phenyl optionally substituted with at least one substituent independently selected from the group consisting of COOH, COO-C₁₋₆ alkyl, C₁₋₆ hydroxyalkyl, -CH₂-R₉.

5. The compound of formula (I) according to claim 1, wherein:
R₃ is selected from the group consisting of H, COOH, COOCH₃, CH₂OH;
R₄ is selected from the group consisting of H, COOH, COOCH₃, CH₂OH; and
R₅ is selected from the group consisting of H, COOH, COOCH₃, CH₂OH.

6. The compound of formula (I) according to claim 1, wherein the compound of formula (I) is selected from the group consisting of: 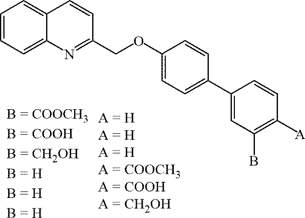 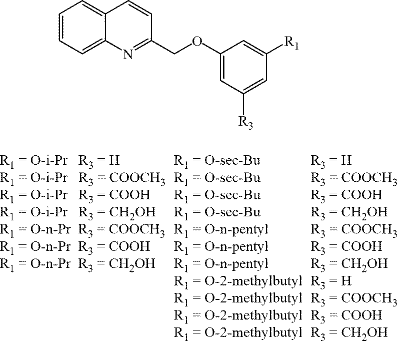 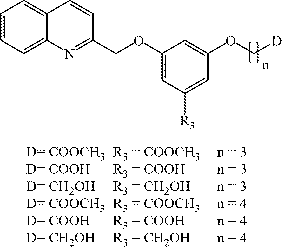 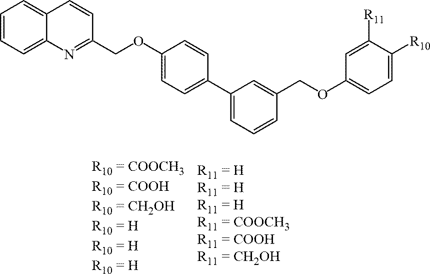 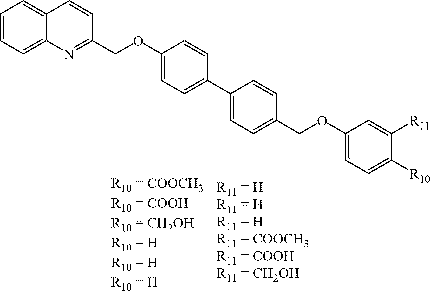 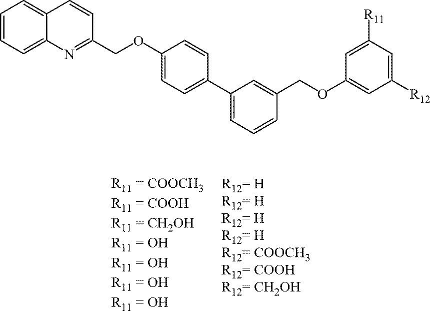 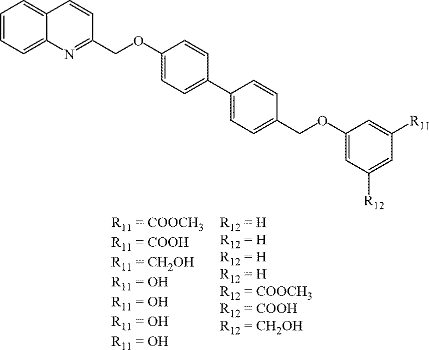 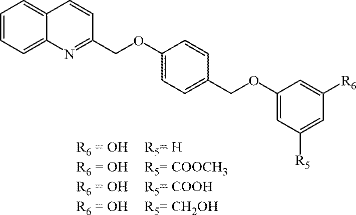 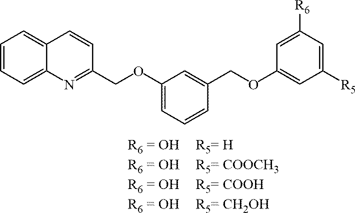 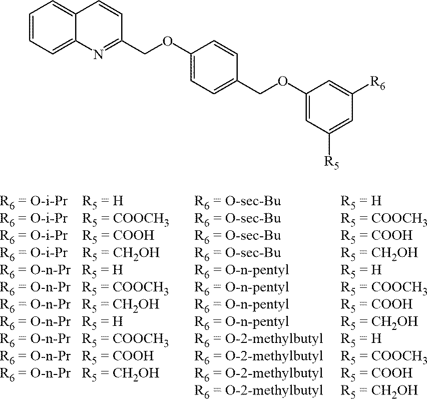 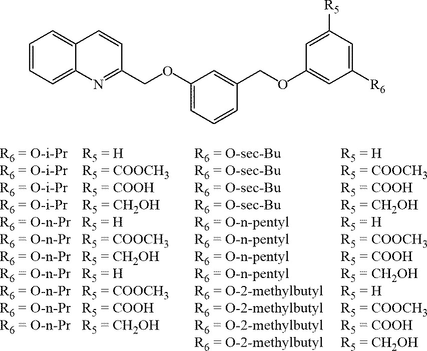 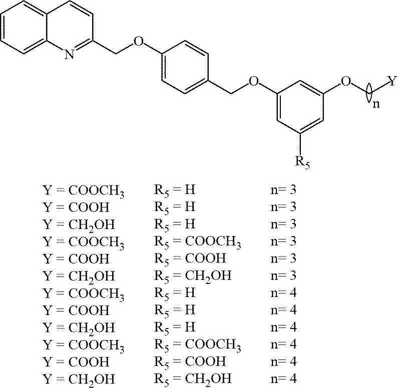 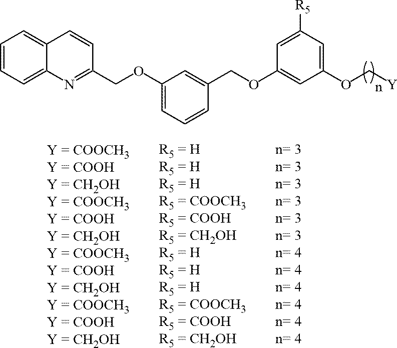
.

7. ( The compound of formula (I) according to claim 6, wherein the compound of formula (I) is selected from the group consisting of: 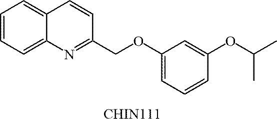 [SMILES: CC(C)Oc1cccc(OCc2ccc3ccccc3n2)c1 ; "CHIN111"] 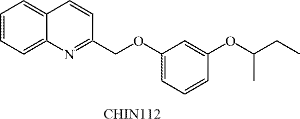 [SMILES: CCC(C)Oc1cccc(OCc2ccc3ccccc3n2)c1 ; "CHIN112"] 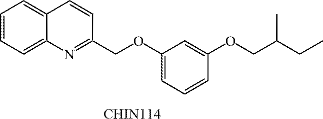 [SMILES: CCC(C)COc1cccc(OCc2ccc3ccccc3n2)c1 ; "CHIN114"] 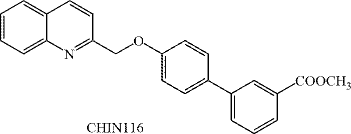 [SMILES: "CHIN116" ; COC(=O)c1cccc(-c2ccc(OCc3ccc4ccccc4n3)cc2)c1] 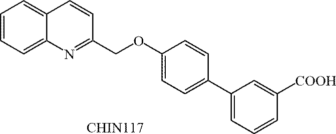 [SMILES: "CHIN117" ; O=C(O)c1cccc(-c2ccc(OCc3ccc4ccccc4n3)cc2)c1] 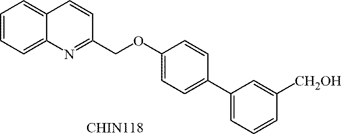 [SMILES: "CHIN118" ; OCc1cccc(-c2ccc(OCc3ccc4ccccc4n3)cc2)c1] 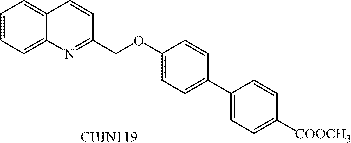 [SMILES: "CHIN119" ; COC(=O)c1ccc(-c2ccc(OCc3ccc4ccccc4n3)cc2)cc1] 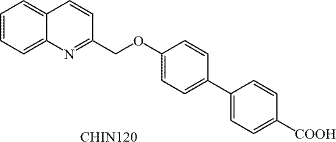 [SMILES: "CHIN120" ; O=C(O)c1ccc(-c2ccc(OCc3ccc4ccccc4n3)cc2)cc1] 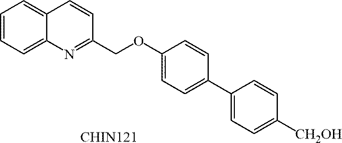 [SMILES: "CHIN121" ; OCc1ccc(-c2ccc(OCc3ccc4ccccc4n3)cc2)cc1] 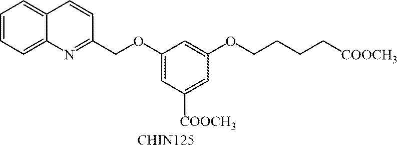 [SMILES: COC(=O)CCCCOc1cc(OCc2ccc3ccccc3n2)cc(C(=O)OC)c1 ; "CHIN125"] 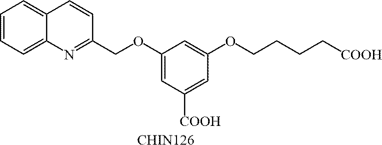 [SMILES: O=C(O)CCCCOc1cc(OCc2ccc3ccccc3n2)cc(C(=O)O)c1 ; "CHIN126"] 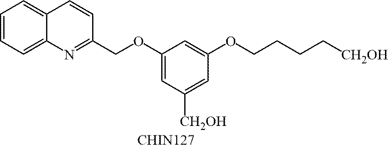 [SMILES: OCCCCCOc1cc(CO)cc(OCc2ccc3ccccc3n2)c1 ; "CHIN127"] 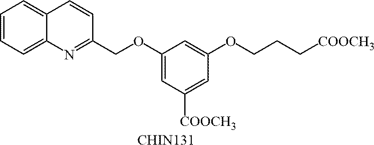 [SMILES: COC(=O)CCCOc1cc(OCc2ccc3ccccc3n2)cc(C(=O)OC)c1 ; "CHIN131"] 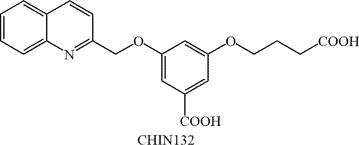 [SMILES: O=C(O)CCCOc1cc(OCc2ccc3ccccc3n2)cc(C(=O)O)c1 ; "CHIN132"] 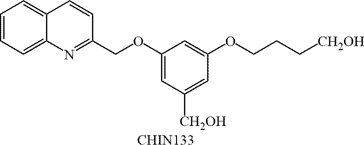 [SMILES: OCCCCOc1cc(CO)cc(OCc2ccc3ccccc3n2)c1 ; "CHIN133"] 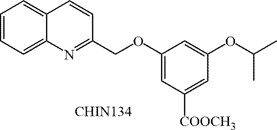 [SMILES: "CHIN134" ; COC(=O)c1cc(OCc2ccc3ccccc3n2)cc(OC(C)C)c1] 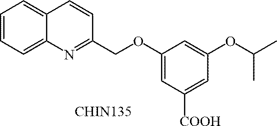 [SMILES: "CHIN135" ; CC(C)Oc1cc(OCc2ccc3ccccc3n2)cc(C(=O)O)c1] 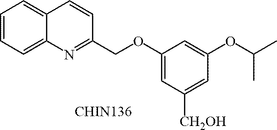 [SMILES: "CHIN136" ; CC(C)Oc1cc(CO)cc(OCc2ccc3ccccc3n2)c1] 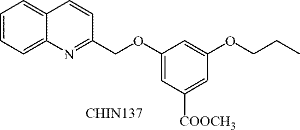 [SMILES: "CHIN137" ; CCCOc1cc(OCc2ccc3ccccc3n2)cc(C(=O)OC)c1] 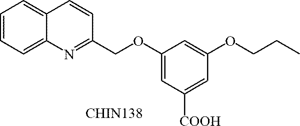 [SMILES: "CHIN138" ; CCCOc1cc(OCc2ccc3ccccc3n2)cc(C(=O)O)c1] 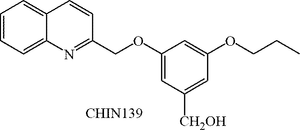 [SMILES: "CHIN139" ; CCCOc1cc(CO)cc(OCc2ccc3ccccc3n2)c1] 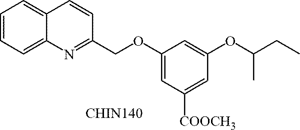 [SMILES: "CHIN140" ; CCC(C)Oc1cc(OCc2ccc3ccccc3n2)cc(C(=O)OC)c1] 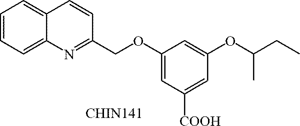 [SMILES: "CHIN141" ; CCC(C)Oc1cc(OCc2ccc3ccccc3n2)cc(C(=O)O)c1] 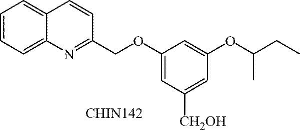 [SMILES: "CHIN142" ; CCC(C)Oc1cc(CO)cc(OCc2ccc3ccccc3n2)c1] 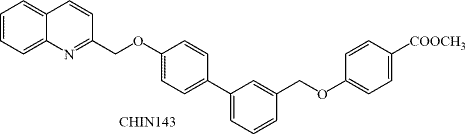 [SMILES: "CHIN143" ; COC(=O)c1ccc(OCc2cccc(-c3ccc(OCc4ccc5ccccc5n4)cc3)c2)cc1] 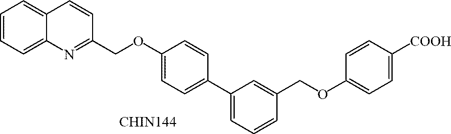 [SMILES: "CHIN144" ; O=C(O)c1ccc(OCc2cccc(-c3ccc(OCc4ccc5ccccc5n4)cc3)c2)cc1] 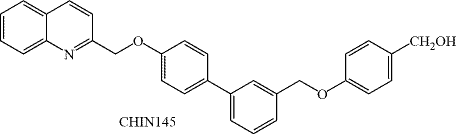 [SMILES: "CHIN145" ; OCc1ccc(OCc2cccc(-c3ccc(OCc4ccc5ccccc5n4)cc3)c2)cc1] 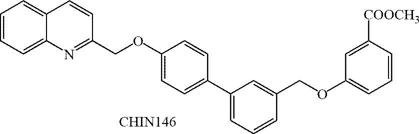 [SMILES: "CHIN146" ; COC(=O)c1cccc(OCc2cccc(-c3ccc(OCc4ccc5ccccc5n4)cc3)c2)c1] 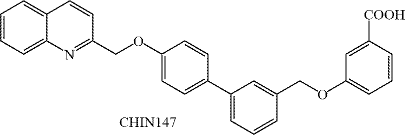 [SMILES: "CHIN147" ; O=C(O)c1cccc(OCc2cccc(-c3ccc(OCc4ccc5ccccc5n4)cc3)c2)c1] 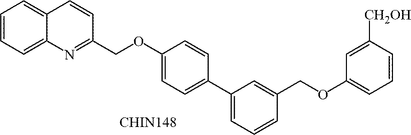 [SMILES: "CHIN148" ; OCc1cccc(OCc2cccc(-c3ccc(OCc4ccc5ccccc5n4)cc3)c2)c1] 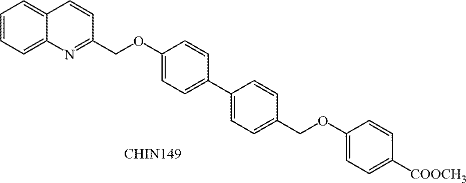 [SMILES: "CHIN149" ; COC(=O)c1ccc(OCc2ccc(-c3ccc(OCc4ccc5ccccc5n4)cc3)cc2)cc1] 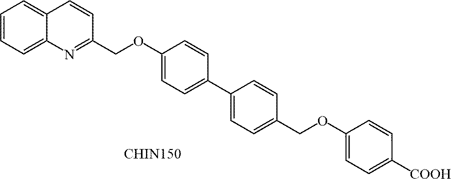 [SMILES: "CHIN150" ; O=C(O)c1ccc(OCc2ccc(-c3ccc(OCc4ccc5ccccc5n4)cc3)cc2)cc1] 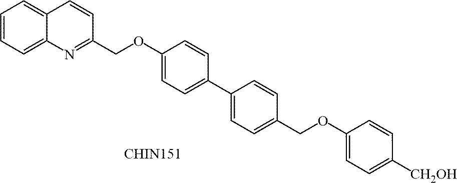 [SMILES: "CHIN151" ; OCc1ccc(OCc2ccc(-c3ccc(OCc4ccc5ccccc5n4)cc3)cc2)cc1] 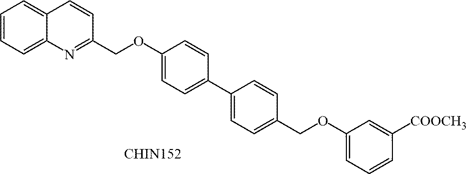 [SMILES: "CHIN152" ; COC(=O)c1cccc(OCc2ccc(-c3ccc(OCc4ccc5ccccc5n4)cc3)cc2)c1] 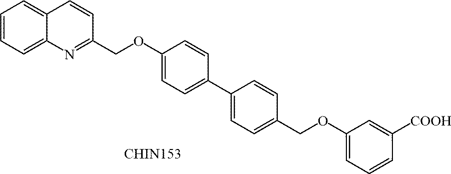 [SMILES: "CHIN153" ; O=C(O)c1cccc(OCc2ccc(-c3ccc(OCc4ccc5ccccc5n4)cc3)cc2)c1] 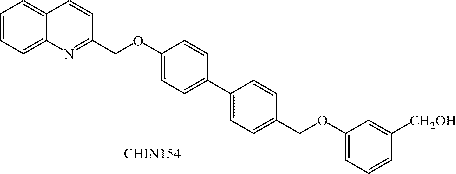 [SMILES: "CHIN154" ; OCc1cccc(OCc2ccc(-c3ccc(OCc4ccc5ccccc5n4)cc3)cc2)c1] 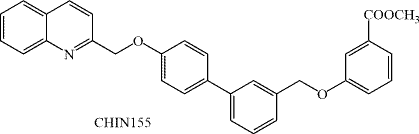 [SMILES: "CHIN155" ; COC(=O)c1cccc(OCc2cccc(-c3ccc(OCc4ccc5ccccc5n4)cc3)c2)c1] 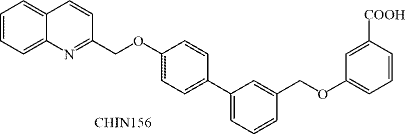 [SMILES: "CHIN156" ; O=C(O)c1cccc(OCc2cccc(-c3ccc(OCc4ccc5ccccc5n4)cc3)c2)c1] 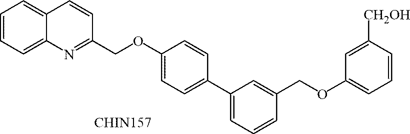 [SMILES: "CHIN157" ; OCc1cccc(OCc2cccc(-c3ccc(OCc4ccc5ccccc5n4)cc3)c2)c1] 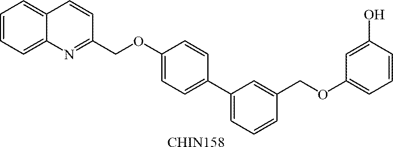 [SMILES: Oc1cccc(OCc2cccc(-c3ccc(OCc4ccc5ccccc5n4)cc3)c2)c1 ; "CHIN158"] 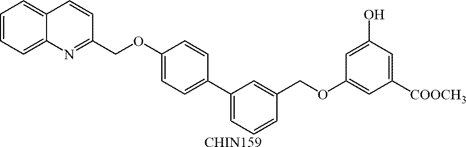 [SMILES: COC(=O)c1cc(O)cc(OCc2cccc(-c3ccc(OCc4ccc5ccccc5n4)cc3)c2)c1 ; "CHIN159"] 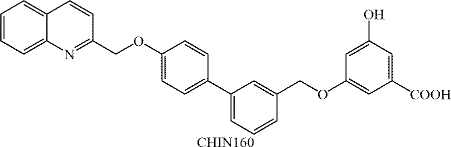 [SMILES: O=C(O)c1cc(O)cc(OCc2cccc(-c3ccc(OCc4ccc5ccccc5n4)cc3)c2)c1 ; "CHIN160"] 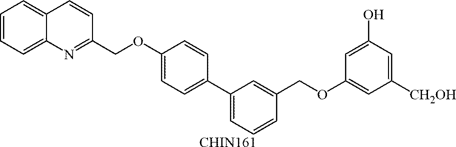 [SMILES: OCc1cc(O)cc(OCc2cccc(-c3ccc(OCc4ccc5ccccc5n4)cc3)c2)c1 ; "CHIN161"] 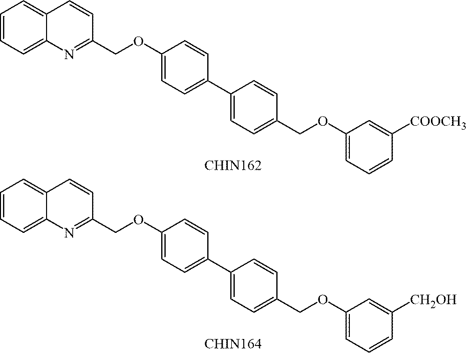 [SMILES: "CHIN162" ; COC(=O)c1cccc(OCc2ccc(-c3ccc(OCc4ccc5ccccc5n4)cc3)cc2)c1 ; "CHIN164" ; OCc1cccc(OCc2ccc(-c3ccc(OCc4ccc5ccccc5n4)cc3)cc2)c1] 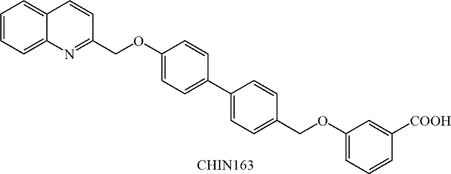 [SMILES: "CHIN163" ; O=C(O)c1cccc(OCc2ccc(-c3ccc(OCc4ccc5ccccc5n4)cc3)cc2)c1] 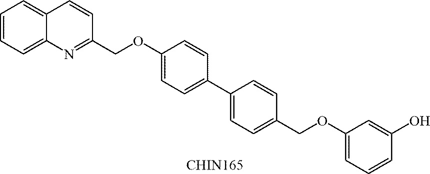 [SMILES: "CHIN165" ; Oc1cccc(OCc2ccc(-c3ccc(OCc4ccc5ccccc5n4)cc3)cc2)c1] 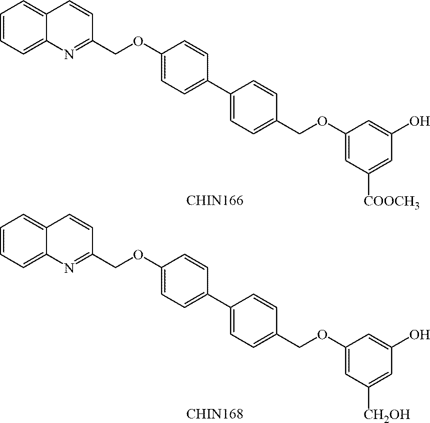 [SMILES: "CHIN166" ; COC(=O)c1cc(O)cc(OCc2ccc(-c3ccc(OCc4ccc5ccccc5n4)cc3)cc2)c1 ; "CHIN168" ; OCc1cc(O)cc(OCc2ccc(-c3ccc(OCc4ccc5ccccc5n4)cc3)cc2)c1] 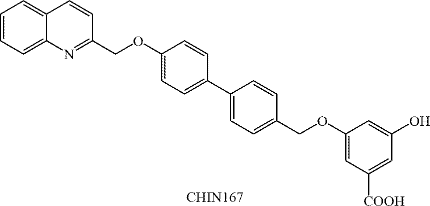 [SMILES: "CHIN167" ; O=C(O)c1cc(O)cc(OCc2ccc(-c3ccc(OCc4ccc5ccccc5n4)cc3)cc2)c1]
.

8. A compound of formula (Ia) 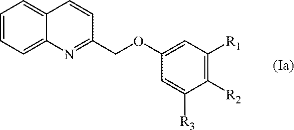
or pharmaceutically acceptable salts or solvates thereof wherein:
R₁ is selected from the group consisting of H, linear or branched C₁₋₆ alkyl optionally substituted with one substituent R₇; linear or branched O-C₃₋₆ alkyl optionally substituted with one substituent R₈;
R₂ is selected from the group consisting of H, C₁₋₆ hydroxyalkyl, linear or branched C₁₋₆ alkyl optionally substituted with one substituent R₇; phenyl optionally substituted with at least one substituent independently selected from the group consisting of H, COOH, COO-C₁₋₆ alkyl, C₁₋₆ hydroxyalkyl and linear or branched C₁₋₆ alkyl optionally substituted with one substituent R₉;
R₃ is selected from the group consisting of H, COOH, COO-C₁₋₆ alkyl, CH₂OH; provided that:
when R₂ is H, R₁ or R₃ are not H; or
when R₁ is linear O-C₃₋₆ alkyl optionally substituted with one substituent R₈, at least one between R₂ and R₃ is not H;
R₄ is selected from the group consisting of H, COOH, COO-C₁₋₆ alkyl, C₁₋₆ hydroxyalkyl;
R₅ is selected from the group consisting of H, COOH, COO-C₁₋₆ alkyl, C₁₋₆ hydroxyalkyl;
R₆ is selected from the group consisting of H, COOH, COO-C₁₋₆ alkyl, C₁₋₆ hydroxyalkyl, OH and linear or branched C₁₋₆ alkoxyl optionally substituted with one substituent R₈;
provided that when one between R₄, R₅ and R₆ is COOH or CH₂OH, at least one of the remaining ones between R₄, R₅ and R₆ is not H,
R₇ is selected from the group consisting of: 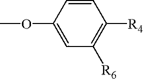 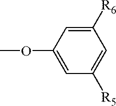
R
₈ is selected from the group consisting of OH, COOH, COO-C₁₋₆ alkyl;
R₉ is selected from the group consisting of: 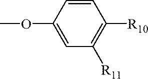 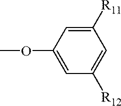
where R
₁₀ is selected from the group consisting of H, COOH, COO-C₁₋₆ alkyl, C₁₋₆ hydroxyalkyl;
R₁₁ is selected from the group consisting of H, OH, COOH, COO-C₁₋₆ alkyl, C₁₋₆ hydroxyalkyl;
and R₁₂ is selected from the group consisting of H, COOH, COO-C₁₋₆ alkyl, C₁₋₆ hydroxyalkyl;
for the use as a medicament.

9. A method for the prevention and/or treatment of a disorder, comprising administering to a subject an effective amount of the compound of claim 8, wherein the disorder is selected from the group consisting of gastrointestinal disorders, liver diseases, cardiovascular diseases, atherosclerosis, metabolic diseases, metabolic disorders, infectious diseases, cancer, renal disorders, inflammatory disorders, and neurological disorders.

10. A pharmaceutical composition comprising a compound of formula (Ia): 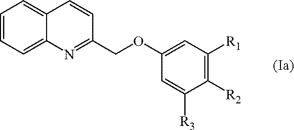
or pharmaceutically acceptable salts or solvates thereof wherein:
R₁ is selected from the group consisting of H, linear or branched C₁₋₆ alkyl optionally substituted with one substituent R₇; linear or branched O-C₃₋₆ alkyl optionally substituted with one substituent R₈;
R₂ is selected from the group consisting of H, C₁₋₆ hydroxyalkyl, linear or branched C₁₋₆ alkyl optionally substituted with one substituent R₇; phenyl optionally substituted with at least one substituent independently selected from the group consisting of H, COOH, COO-C₁₋₆ alkyl, C₁₋₆ hydroxyalkyl and linear or branched C₁₋₆ alkyl optionally substituted with one substituent R₉;
R₃ is selected from the group consisting of H, COOH, COO-C₁₋₆ alkyl, CH₂OH; provided that:
when R₂ is H, R₁ or R₃ are not H; or
when R₁ is linear O-C₃₋₆ alkyl optionally substituted with one substituent R₈, at least one between R₂ and R₃ is not H;
R₄ is selected from the group consisting of H, COOH, COO-C₁₋₆ alkyl, C₁₋₆ hydroxyalkyl;
R₅ is selected from the group consisting of H, COOH, COO-C₁₋₆ alkyl, C₁₋₆ hydroxyalkyl;
R₆ is selected from the group consisting of H, COOH, COO-C₁₋₆ alkyl, C₁₋₆ hydroxyalkyl, OH and linear or branched C₁₋₆ alkoxyl optionally substituted with one substituent R₈;
provided that when one between R₄, R₅ and R₆ is COOH or CH₂OH, at least one of the remaining ones between R₄, R₅ and R₆ is not H,
R₇ is selected from the group consisting of: 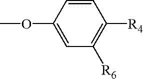 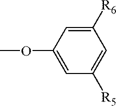
R
₈ is selected from the group consisting of OH, COOH, COO-C₁₋₆ alkyl;
R₉ is selected from the group consisting of: 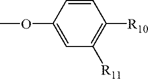 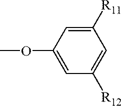
where R
₁₀ is selected from the group consisting of H, COOH, COO-C₁₋₆ alkyl, C₁₋₆ hydroxyalkyl;
R₁₁ is selected from the group consisting of H, OH, COOH, COO-C₁₋₆ alkyl, C₁₋₆ hydroxyalkyl;
and R₁₂ is selected from the group consisting of H, COOH, COO-C₁₋₆ alkyl, C₁₋₆ hydroxyalkyl;
and at least a pharmaceutically acceptable excipient.
